(19) United States
(12) Patent Application Publication
(10) Pub. No.: US 2021/0379574 A1
(43) Pub. Date: Dec. 9, 2021

(54) MULTI-FUNCTIONALIZED HOLLOW FIBER ORGANOCATALYSTS

(71) Applicant: The Curators of the University of Missouri, Columbia, MO (US)

(72) Inventors: Ali A. Rownaghi, Rolla, MO (US); Fateme Rezaei, Rolla, MO (US); Abdo-Alslam Alwakwak, Rolla, MO (US)

(21) Appl. No.: 17/340,658

(22) Filed: Jun. 7, 2021

Related U.S. Application Data
(60) Provisional application No. 63/036,090, filed on Jun. 8, 2020.

(51) Int. Cl. B01J 31/12; B01J 37/04; B01J 37/06; B01J 37/08; B01J 35/10; B01J 8/02; C07D 263/22; C07C 213/04; D01D 5/24; D01F 11/00

(52) U.S. Cl. CPC B01J 31/123; B01J 37/04; B01J 37/06; B01J 37/08; B01J 35/1014; D01F 11/00; B01J 8/0221; C07D 263/22; C07C 213/04; D01D 5/24; B01J 35/1038

(57) ABSTRACT

Described herein are multi-functionalized hollow fiber organocatalysts, processes for producing multi-functionalized hollow fiber organocatalysts, and processes that utilize multi-functionalized hollow fiber organocatalysts for reacting chemicals. A variety of chemical reactions may be enhanced with the multifunctional hollow fiber organocatalysts. The multi-functionalized hollow fiber organocatalysts are particularly advantageous when used as heterogeneous organocatalysts and continuous-flow reactors.

### CROSS-REFERENCE TO RELATED APPLICATIONS

This application claims priority to U.S. Provisional Application Ser. No. 63/036,090, filed on Jun. 8, 2020, the content of which is hereby incorporated by reference in its entirety.

### FIELD OF THE DISCLOSURE

Described herein are multi-functionalized hollow fiber organocatalysts, processes for producing multi-functionalized hollow fiber organocatalysts, and processes that utilize multi-functionalized hollow fiber organocatalysts for reacting chemicals. A variety of chemical reactions may be enhanced with the multifunctional hollow fiber organocatalysts. The multi-functionalized hollow fiber organocatalysts are particularly advantageous when used as heterogeneous organocatalysts and continuous-flow reactors.

### BACKGROUND OF THE DISCLOSURE

The use of multifunctional organocatalysts and continuous flow platforms are commonplace in modern chemical transformation and attract a great deal of interest with respect to economic and environmentally-sustainable production of pharmaceuticals, fine chemicals, and agrochemicals, water treatment, as well as upgrading of biomass feedstocks. For example, the conversion of CO₂ into organic carbonates for the chemical/process industry is an efficient way to rapidly introduce renewable feedstocks in this value chain. The catalytic cycloaddition of CO₂ on epoxides still represents the most important route for the synthesis of cyclic carbonates that are intermediates for the further hydroxyalkylation of amines (aminolysis) and formation of aminoalcohols. The major concern of this reaction is from both safety and environmental standpoints because epoxides are extremely flammable, highly toxic, and carcinogenic compounds. These issues have created a long-standing interest in sustainable chemical transformation promoted by organic carbonates as a valuable option for hydroxyalkylation reactions of amines because cyclic carbonates are non-toxic products and not flammable.

Aminoalcohols are important organic intermediates in the synthesis of organic compounds, amino acids, pharmaceutical compounds and play an important role as chiral auxiliaries and chiral ligands in asymmetric catalysis. Both CO₂ cycloaddition and hydroxyalkylation reactions of amines have been studied over both homogeneous and heterogeneous catalysts such as organocatalysts, Lewis acids and Lewis bases, and organic/inorganic salts such as metal triflates, metal halides, N-heterocyclic carbenes, and β-cyclodextrin. Most cooperative organocatalysts for CO₂ cycloaddition have been based on silica-supported amines.

Ionic liquids (ILs) have been identified to be sustainable solvents and organocatalysts for a number of chemical transformations such as CO₂ activation and cyanosilylation of aldehydes/ketones and various carbonyl compounds. For example, the reaction of primary aromatic amines pX-C₆H₄NH₂; (X=H, OCH₃, CH₃, Cl) with ethylene- and propylene-carbonates for the selective synthesis of bis-N-(2-hydroxy) alkylanilines over a class of phosphonium ionic liquids (PILs) such as tetraalkylphosphonium halides, and tosylates turn out to be active organocatalysts for both aniline and other primary aromatic amine hydroxyalkylation at 140° C. under solvent-less conditions. A kinetic analysis confirmed that bromide exchanged PILs are the most efficient systems, able to impart a more than 8-fold acceleration to the reaction. Metal-free synthesis of N-aryl-carbamates from cyclic organic carbonates and aromatic amines over triazabicyclodecene organocatalyst has also been studied.

During a homogeneous hydroxyalkylation reaction, catalyst deactivation occurs, which necessitates frequent catalyst regeneration. Another shortcoming is that in conventional batch and plug-flow systems, the reagents and homogeneous catalyst are flowed through the reactor together. Thus, separation of the product from the catalyst and possible byproducts is required. The improvement of catalyst lifetime is one of the key challenges in homogeneous catalyst development. Thus, it is necessary to develop new catalytic methodologies that offer simpler and more cost-effective approaches for permanent immobilization of homogeneous catalysts on solid surface to replace homogeneous stoichiometric reagents for sustainable chemical transformation. In this regard, porous polymers are relatively inexpensive and versatile materials that can be utilized as an organocatalyst support. Furthermore, metal nanoparticles have been encapsulated by using ionic liquids as stabilizing agents in various porous polymers such as porous cellulose, polysulfone, polyvinylidene fluoride, polythiosemicarbazide, and polythiourea, and have demonstrated that such heterogeneous catalytic system can be used in cross-coupling reactions, hydrogenation, and treatment of diatrizoate-contaminated water. Immobilization of multifunctional organocatalysts upon porous polymer surfaces can be tuned by controlling the linker length, ratio of the active spices and controlling the support pore size.

Current aminoalcohol production uses batch reactor technology for this process; however, it is unfavorable for industrial applications compared to flow processes due to mass and heat transfer limitations, time and energy-consumption for the reaction, and subsequent purification of product and catalyst steps. Substitution of batch processes by flow processes can contribute to the implementation of the sustainability of chemical transformation through process intensification. In general, process intensification seems to be a more sustainable approach than an analogous batch due to integration of multifunctional reactors and transport processes in one vessel to improve the control over reaction performance, achieve higher yield, and to make the process more efficient to save operational and capital costs. The characteristic properties of microstructured reactors are their ultrafast heat and mass transfer and unique way to perform exothermic reactions. As such, the application of new synthetic catalytic technologies can play a central role in the production of fine chemicals and pharmaceuticals. However, the major drawback of the flow process is the decomposition and leaching of the active species from the support and their subsequent transport to the product stream. This ultimately leads to significant catalyst loss and product contamination. Catalyst leaching can be critical, even in very small quantities, in product solution and last it can further catalyze chemical reactions with surprisingly high efficiency. Current systems do not address the issue of permanent immobilization of the organocatalyst into various flow systems.

Continuous flow chemistry is a process in which reactants are mixed and react together in flow reactor by under precisely controlled reaction conditions. Immobilization of cooperative organocatalysts such as acid-base bifunctional catalysts, and metal nanoparticles such as palladium, copper, ruthenium, and nickel, within continuous-flow microreactors offers an efficient catalytic system that exploits and enhances the advantages of both nanocatalysis and flow chemistry, the so-called flow nanocatalysis approach. Various strategies have been developed for implementation of continuous-flow reactions including the immobilization and subsequent anchoring of catalysts within microstructured reactors. However, many applications of continuous-flow reactions are still hampered by linkers/supports decomposition and catalysts subsequent leaching from the reactor to the product stream. In addition, most flow reactors have undertaken approaches inspired by earlier continuous packed-bed reactor studies and do not address issues of permanent immobilization of catalyst species, economic feasibility, and scalability.

Immobilizing organocatalysts into a hollow fiber microfluidic reactor matrix is an effective approach for overcoming the aforementioned drawbacks related to catalyst deactivation and for non-leaching catalysis. Since catalyst leaching is avoided, diffusion limitations are reduced and selective removal of the products can shift the equilibrium state of the reaction and avoid side reactions. To date, a simple and convenient method for permanent immobilization of multifunctional organocatalysts within porous polymer for continuous-flow reaction that addresses stability, reactivity and recyclability of the obtained catalyst has not yet been established. A metal-free and sustainable process would represent a valuable alternative to metal-based processes which require either harsh reaction conditions or more expensive reagents and/or metal precursors.

To address the above issues, described herein is an asymmetric porous polymer hollow fiber microfluidic reactor that combines both immobilized organocatalysts and final product formulations into a single, highly compact unit. The hierarchical pore structure networks of porous hollow fiber polymer are used to immobilize a variety of organocatalyst active species, thus leading to catalyze numerous reaction steps in a continuous flow platform. The catalysts of the present disclosure have wide technological and commercial applications.

### BRIEF DESCRIPTION OF THE DISCLOSURE

In one embodiment, the present disclosure is directed to an organocatalyst comprising a porous hollow fiber and at least two functional groups covalently bonded to the porous hollow fiber, wherein the organocatalyst is free of metals.

In another embodiment, the present disclosure is directed to a process for producing an organocatalyst comprising a porous hollow fiber and at least two functional groups covalently bonded to the porous hollow fiber, wherein the organocatalyst is free of metals, the process comprising (i) spinning a porous hollow fiber, (ii) grafting a first functional group to the porous hollow fiber, and (iii) grafting a second functional group to the porous hollow fiber.

In yet another embodiment, the present disclosure is directed to a process for reacting chemicals, the process comprising (i) introducing a first reactant to an organocatalyst comprising a porous hollow fiber and at least two functional groups covalently bonded to the porous hollow fiber, wherein the organocatalyst is free of metals, (ii) introducing a second reactant to the organocatalyst, and (iii) reacting the first reactant and the second reactant.

### BRIEF DESCRIPTION OF THE DRAWINGS

FIG. 1 is an exemplary embodiment of a schematic diagram of a microfluidic reactor containing multi-functionalized hollow fiber organocatalysts in accordance with the present disclosure.

FIG. 2 is an exemplary embodiment of a schematic diagram of a polyamide-imide hollow fiber spinning apparatus in accordance with the present disclosure.

FIG. 3 is an exemplary embodiment of a process flow diagram of a reactor setup in accordance with the present disclosure.

FIG. 4 depicts exemplary embodiments of nitrogen adsorption/desorption isotherms before and after reaction for bare PAIHFs, APS/PAIHF and Br/APS/PAIHF in accordance with the present disclosure.

FIG. 5 depicts exemplary embodiments of XPS spectra before and after reaction of bare PAIHFs, APS/PAIHFs, Br/APS/PAIHFs in accordance with the present disclosure.

FIG. 6 depicts exemplary embodiments of IR spectra before and after reaction of bare PAIHF, APS/PAIHF and Br/APS/PAIHF in accordance with the present disclosure.

FIG. 7A depicts an exemplary embodiment of an SEM image of the cross section of a Br/APS/PAIHF in accordance with the present disclosure.

FIG. 7B depicts an exemplary embodiment of an SEM image of the surface of the Br/APS/PAIHF (before reaction) in accordance with the present disclosure.

FIG. 7C depicts an exemplary embodiment of an SEM image of the surface of the Br/APS/PAIHF (after reaction) in accordance with the present disclosure.

FIG. 8A depicts an exemplary embodiment of the effects of different CO₂ pressures on CO₂ cycloaddition of propylene oxide to propylene carbonate at 140° C., during a 1 hour reaction time, in accordance with the present disclosure.

FIG. 8B depicts an exemplary embodiment of the effects of 5 bar CO₂ pressure and different reaction times on CO₂ cycloaddition of propylene oxide to propylene carbonate at 140° C. in accordance with the present disclosure.

FIG. 8C depicts an exemplary embodiment of the effects of tandem reaction of CO₂ cycloaddition and hydroxyalkylation of aniline at different reaction times (5 bar CO₂ pressure, at 140° C.) on CO₂ cycloaddition of propylene oxide to propylene carbonate at 140° C. in accordance with the present disclosure.

FIG. 9A depicts an exemplary embodiment of the effects of different reaction times and ambient pressures on the reaction of propylene carbonate (PC) with aniline (A) to phenylamino)propan-2-ol (AA) at 140° C. in accordance with the present disclosure.

FIG. 9B depicts an exemplary embodiment of the effects of various catalyst loadings on the reaction of propylene carbonate (PC) with aniline (A) to phenylamino)propan-2-ol (AA) at 140° C. during a 1 hour reaction time in accordance with the present disclosure.

FIG. 10A depicts an exemplary embodiment of the effects of flow rate on aniline conversion and selectivity in hydroxyalkylation of aniline with propylene carbonate at 140° C. and ambient pressure for 0.02 cm³/min in accordance with the present disclosure.

FIG. 10B depicts an exemplary embodiment of the effects of flow rate on aniline conversion and selectivity in hydroxyalkylation of aniline with propylene carbonate at 140° C. and ambient pressure for 0.04 cm³/min in accordance with the present disclosure.

FIG. 10C depicts an exemplary embodiment of the effects of flow rate on aniline conversion and selectivity in hydroxyalkylation of aniline with propylene carbonate at 140° C. and ambient pressure for 0.08 cm³/min in accordance with the present disclosure.

FIG. 11A depicts an exemplary embodiment of N₂ physisorption isotherms for HF-PAI (bare), HF-PAI/APS, HF-PAI/APS/Na-2-Br, and HF-PAI/APS/3-Br-Prop in accordance with the present disclosure.

FIG. 11B depicts an exemplary embodiment of pore size distributions for HF-PAI (bare), HF-PAI/APS, HF-PAI/APS/Na-2-Br, and HF-PAI/APS/3-Br-Prop in accordance with the present disclosure.

FIG. 12A depicts an exemplary embodiment of N2 physisorption isotherms for HF-PAI (bare), HF-PAI/APS, and HF-PAI/APS/F in accordance with the present disclosure.

FIG. 12B depicts an exemplary embodiment of pore size distributions for HF-PAI (bare), HF-PAI/APS, and HF-PAI/APS/F in accordance with the present disclosure.

FIG. 13A depicts an exemplary embodiment of IR spectra of bare [HF-PAI], [HF-PAI/APS], and [HF-PAI/APS/3-Br-Prop] in accordance with the present disclosure.

FIG. 13B depicts an exemplary embodiment of an IR spectrum of [HF-PAI/APS/3-Br-Prop] in accordance with the present disclosure.

FIG. 14A depicts an exemplary embodiment of IR spectra of bare [HF-PAI], [HF-PAI/APS], and [HF-PAI/APS/Na-2-Br] in accordance with the present disclosure.

FIG. 14B depicts an exemplary embodiment of an IR spectrum of [HF-PAI/APS/Na-2-Br] in accordance with the present disclosure.

FIG. 15A depicts an exemplary embodiment of IR spectra of bare [HF-PAI], [HF-PAI/APS], and [HF-PAI/APS/F] in accordance with the present disclosure.

FIG. 15B depicts an exemplary embodiment of an IR spectrum of [HF-PAI/APS/F] in accordance with the present disclosure.

FIG. 16A depicts an exemplary embodiment of an SEM image of the cross section of bare HF-PAI in accordance with the present disclosure.

FIG. 16B depicts an exemplary embodiment of an SEM image of the surface of bare HF-PAI in accordance with the present disclosure.

FIG. 16C depicts an exemplary embodiment of an SEM image of the surface of the HF-PAI/APS in accordance with the present disclosure.

FIG. 16D depicts an exemplary embodiment of an SEM image of the surface of the HF-PAI/APS/Br in accordance with the present disclosure.

FIG. 16E depicts an exemplary embodiment of an SEM image of the surface of the HF-PAI/APS/Br (after reaction) in accordance with the present disclosure.

FIG. 17A depicts an exemplary embodiment of the effects of reaction time on the butyl amine conversion and the selectivity of [1-(butylamino)] propane-2-ol in the presence of HF33-PAI/APS/Na-2-Br in accordance with the present disclosure.

FIG. 17B depicts an exemplary embodiment of the effects of reaction time on the butyl amine conversion and the selectivity of [1-(butylamino)] propane-2-ol in the presence of HF33-PAI/APS/3-Br-Prop in accordance with the present disclosure.

FIG. 18A depicts an exemplary embodiment of the effects of reaction time on the butyl amine conversion and the selectivity of 1-(hexylamino) propan-2-ol in the presence of HF33-PAI/APS/Na-2-Br in accordance with the present disclosure.

FIG. 18B depicts an exemplary embodiment of the effects of reaction time on the butyl amine conversion and the selectivity of 1-(hexylamino) propan-2-ol in the presence of HF33-PAI/APS/3-Br-Prop in accordance with the present disclosure.

FIG. 19A depicts an exemplary embodiment of the effects of reaction time on aniline conversion and the selectivity of 1-(phenylamino) propan-2-ol in the presence of HF-PAI/APS/Na-2-Br in accordance with the present disclosure.

FIG. 19B depicts an exemplary embodiment of the effects of reaction time on aniline conversion and the selectivity of 1-(phenylamino) propan-2-ol in the presence of HF-PAI/APS/3-Br-Prop in accordance with the present disclosure.

FIG. 20 depicts an exemplary embodiment of the effects of reaction time on 4-aminophenol conversion and the selectivity of 4-[(2-hydroxypropyl) amino]- in the presence of HF-PAI/APS/Na-2-Br and HF-PAI/APS/3-Br-Prop in accordance with the present disclosure.

FIG. 21 depicts an exemplary embodiment of the effects of reaction time on aniline conversion and the selectivity of 1-(phenylamino) propan-2-ol in the presence of HF-PAI/APS/F in accordance with the present disclosure.

FIG. 22 depicts an exemplary embodiment of the effects of reaction time on 4-aminophenol conversion and the selectivity of 4-[(2-hydroxypropyl) amino]- in the presence of HF33-PAI/APS/F in accordance with the present disclosure.

FIG. 23 depicts an exemplary embodiment of the effects of reaction time on hexyl amine conversion and the selectivity of 1-(hexylamino) propan-2-ol in the presence of HF33-PAI/APS/F in accordance with the present disclosure.

### DETAILED DESCRIPTION OF THE DISCLOSURE

Described herein are a variety of chemical reactions that have been enhanced with multifunctional hollow fiber organocatalysts. Enhancements of the present disclosure include increased efficiency, reaction rates, selectivity, conversion, and processability.

In particular, continuous-flow reactions catalyzed with the multifunctional hollow fiber organocatalysts in accordance with the present disclosure enable process intensification (e.g. waste minimization and cost/energy reduction) to provide a sustainable method for chemical transformation and biomass upgrading. Continuous-flow mode and no back-mixing of the conventional tubular reactor strongly suppress competing side reactions.

Covalent immobilization of cooperative organocatalysts such as hydrogen-bond donor groups (e.g. —OH and —NH), and nucleophilic species upon the fiber surface offers an efficient catalytic system that exploits and enhances the advantages of both nanocatalysis and flow chemistry.

In some embodiments, an organocatalyst in accordance with the present disclosure comprises a porous hollow fiber and at least two functional groups covalently bonded to the porous hollow fiber, where the organocatalyst is free of metals.

In some embodiments, the organocatalyst being free of metals means that the organocatalyst does not comprise a metal selected from the group consisting of metal atoms, compounds comprising metal atoms, and combinations thereof. In many embodiments, metal atoms include noble metals, transition metals, alkali and alkaline earth metals, and lanthanide metals. In some embodiments, metal atoms include noble metals, transition metals, and lanthanide metals. In some embodiments, the organocatalyst is free of metals, meaning that there are 0.00% of metals in the organocatalyst. In some embodiments, the organocatalyst is essentially free of metals, meaning that the organocatalyst comprises less than 1.0%, less than 0.5%, less than 0.1%, less than 0.01% or less than 0.001% of metals.

In some embodiments, the organocatalyst is a cooperative multi-functionalized hollow fiber organocatalyst. In some embodiments, the organocatalyst is a multi-functionalized composite hollow fiber organocatalyst. In some embodiments, the organocatalyst is a cooperative multi-functionalized composite hollow fiber organocatalyst.

In some embodiments, the organocatalyst is a covalent organocatalyst. In some embodiments, the organocatalyst is a non-covalent organocatalyst. In some embodiments, the organocatalyst is selected from the group consisting of covalent organocatalysts, non-covalent organocatalysts, and combinations thereof.

In some embodiments, the organocatalyst comprises a counterion. In some embodiments, the organocatalyst comprises a counterion selected from the group consisting of alkali metals (e.g., Na, K), alkaline metals (e.g., Mg, Ca, Sr), noble metals (e.g., palladium, platinum, gold, rhodium, ruthenium), rare earth metals (such as lanthanum, cerium), and combinations thereof.

In some embodiments, the porous hollow fiber comprises a material selected from the group consisting of polyamide, polyimide, fluorinated polyimide, polyvinylpyrrolidone, polysulfone, porous cellulose, polyvinylidene fluoride, polythiosemicarbazide, polythiourea, polyethylene, polypropylene, polytetrafluoroethylene, fluoropolymers, carbon molecular sieves derived from one or more polymers, zeolite, metal organic frameworks (MOFs), covalent organic framework (COF) nanocrystals incorporated within a polymer mixed-matrix, and combinations thereof.

In some embodiments, asymmetric hollow fiber polymers have a range of microstructural properties. These microstructural properties may be tuned to achieve desired catalytic properties.

In some embodiments, an inner diameter of the asymmetric hollow fiber polymers is in the range of from about 50 μm to about 200 μm. In some embodiments, an inner diameter of the asymmetric hollow fiber polymers is in the range of from about 60 μm to about 120 μm.

In some embodiments, a wall thickness of the asymmetric hollow fiber polymers is in the range of from about 50 μm to about 500 μm. In some embodiments, a wall thickness of the asymmetric hollow fiber polymers is in the range of from about 50 μm to about 100 μm.

In some embodiments, a porosity of the asymmetric hollow fiber polymers is in the range of from about 0.2 to about 1.0. In some embodiments, a porosity of the asymmetric hollow fiber polymers is in the range of from about 0.4 to about 0.6.

In some embodiments, an average pore size of the asymmetric hollow fiber polymers is in the range of from about 50 nm to about 1000 nm. In some embodiments, an average pore size of the asymmetric hollow fiber polymers is in the range of from about 100 nm to about 500 nm.

In some embodiments, a surface roughness of the asymmetric hollow fiber polymers is in the range of from about 5 nm to about 500 nm. In some embodiments, a surface roughness of the asymmetric hollow fiber polymers is in the range of from about 5 nm to about 10 nm.

In some embodiments, the organocatalyst is solvent-free. Solvent-free embodiments are particularly advantageous for industrial-scale and sustainable chemical reactions. In some embodiments, the organocatalyst is essentially free of solvents, meaning that the organocatalyst comprises less than 1.0%, less than 0.5%, less than 0.1%, less than 0.01% or less than 0.001% of solvents.

In some embodiments, the organocatalyst comprises a solvent. In some embodiments, the organocatalyst comprises a solvent selected from the group consisting of polar solvents, non-polar solvents, and combinations thereof. In some embodiments, the organocatalyst comprises a solvent selected from the group consisting of toluene, methanol, ethaol, water, dimethyl sulfoxide (DMSO), dimethyl formamide (DMF), N-methylpyrrolidone (NMP), and combinations thereof.

Generally, multi-functional catalysts refer to groups of multiple, complementary chemical units within close proximity within a single catalytic system that gives rise to their unrivaled chemical rate enhancement. This approach is a simple and scalable synthesis for the creation of a multifunctional, synthetic self-assembled catalytic system. The polyamide imide hollow fibers (PAIHFs) are functionalized with aminosilanes and bromine source to immobilize covalently hydrogen-bond donor groups (—OH and —NH), and nucleophilic [Br⁻] species upon the fiber surface and provide trifunctional acid-base-nucleophilic organocatalysts and microfluidic reactors. The most common multi-functional organocatalysts disclosed herein are those that have acid-base-nucleophilic functions, and a donor group of a hydrogen bond in a suitable position above the chiral scaffold catalyst system. This is also the concept of the catalytic mechanisms that has been applied in the multifunctional hollow fiber microfluidic catalyst reactor system disclosed herein.

In some embodiments, at least one functional group of the at least two functional groups covalently bonded to the porous hollow fiber is a cooperating functional group. A cooperating functional group is a functional group that aids in the localization of the reactant in relation to the porous hollow fiber.

In some embodiments, at least one functional group of the at least two functional groups covalently bonded to the porous hollow fiber is obtained from a source selected from the group consisting of polyamide-imide, 3-aminopropyltrimethoxysilane (APS), 1,3-dibromopropane, sodium 3-bromopropanesulfonate, sodium 2-bromoethanesulfonate, nonafluorobutane-1-sulfonic acid, and combinations thereof.

In some embodiments, post-functionalization of hollow fibers is carried out via click chemistry by using a functional initiator. Post-functionalization with click chemistry allows expansion of the scope of heterogeneous organocatalysts and continuous-flow for chemical applications, biomedical application, and other applications. Functionalized hollow fiber polymers may be obtained without isolation.

In some embodiments, at least one functional group of the at least two functional groups covalently bonded to the porous hollow fiber is a reactive functional group. In some embodiments, at least one functional group of the at least two functional groups covalently bonded to the porous hollow fiber is a non-reactive functional group. The multifunctional groups can act together to undergo cooperative catalysis, which is not possible with only one monofunctional catalyst or bare hollow fibers. Multifunctional hollow fiber organocatalysts are capable of accelerating reactions through cooperative interactions between accurately positioned functional groups present in their active sites. Substrate recognition and activation may proceed through electrostatic, hydrogen bonding, covalent bonding, and combinations thereof.

In some embodiments, the number of functional groups covalently bonded on the organocatalyst is such that that the combined functionalities imparted by the functional groups enhance catalysis of a chemical reaction in a desirable manner. In some embodiments, the functional groups are covalently bonded on the surface of the porous hollow fiber polymer. In some embodiments, the organocatalyst comprises at least three functional groups. In some embodiments, the organocatalyst comprises at least four functional groups. In some embodiments, the organocatalyst comprises at least five functional groups.

In some embodiments, at least one functional group of the at least two functional groups covalently bonded to the porous hollow fiber is bonded covalently in a location of the porous hollow fiber selected from the group consisting of in a pore, on a surface, on an edge, and combinations thereof. In some embodiments, at least one functional group of the at least two functional groups covalently bonded to the porous hollow fiber is bonded covalently in a pore of the porous hollow fiber. In some embodiments, at least one functional group of the at least two functional groups covalently bonded to the porous hollow fiber is bonded covalently on a surface of the porous hollow fiber.

In some embodiments, the cooperative catalysis of heterogeneous hollow fiber organocatalysts is based on the cooperative action of acid-base, acid-thiol, amine-urea, acid-base-halogen, hydroxyl-amine-halogen, imidazole-alcohol-carboxylate groups, and combinations thereof.

In some embodiments, the organocatalyst is a biocatalyst and/or bioactive molecule, such as an enzyme or a protein. Continuous-flow chemistry offers advantages for biocatalysis, avoiding the need for catalyst removal from the product stream, process issues caused by substrate/product inhibition, and equilibrium controlled limitations on yield and allosteric control. In some embodiments, the organocatalyst is a biocatalyst selected from the group consisting of enzymes, cofactors, and combinations thereof. Enzymes in continuous-flow reactors benefit from the advantages of continuous-flow chemistry (i.e. flexibility, control, product stream purity, low capital cost, improved yields for some reactions, etc.).

In some embodiments, at least one functional group of the at least two functional groups covalently bonded to the porous hollow fiber imparts more than one functionality to the organocatalyst. In some embodiments, at least one functional group of the at least two functional groups covalently bonded to the porous hollow fiber imparts more than two functionalities to the organocatalyst. In some embodiments, at least one functional group of the at least two functional groups covalently bonded to the porous hollow fiber imparts more than three functionalities to the organocatalyst.

In some embodiments, at least one functional group of the at least two functional groups covalently bonded to the porous hollow fiber imparts a functionality selected from the group consisting of hydrogen donation, electron donation, electron withdrawal, acidity, basicity, nucleophilicity, electrophilicity, enantioselectivity, and combinations thereof.

In some embodiments, at least one functional group of the at least two functional groups covalently bonded to the porous hollow fiber comprises a functional group selected from the group consisting of halogen, fluorine, chlorine, bromine, iodine, haloalkyl, hydroxyl, sulfur, sulfur trioxide, oxygen, amine, amide, carbonyl, carboxyl, ester, ether, alkane, alkene, alkyne, imidazole, chiral cyclophosphazane, phosphorus amide, silane, aminosilane, and combinations thereof.

In some embodiments, at least one functional group of the at least two functional groups covalently bonded to the porous hollow fiber comprises a functional group selected from the group consisting of halogen, fluorine, chlorine, bromine, iodine, haloalkyl, hydroxyl, amine, silane, aminosilane, sulfur trioxide, and combinations thereof.

In some embodiments, the organocatalyst comprises a halogen group and an aminosilane group. In some embodiments, the organocatalyst is selected from the group consisting of HF-PAI, HF-PAI/APS, HF-PAI/APS/Br, HF-PAI/APS/3-Br-Prop, HF-PAI/APS/F, HF-PAI/APS/Na-2-Br, and combinations thereof.

In some embodiments, the organocatalyst comprises hollow fibers treated with a surface modification process to functionalize with 3-aminopropyl trimethoxy silane (APS). This step can graft the amino group as an APS layer on the surface of the porous hollow fibers and provide a weak-base characteristic. Meanwhile, the combination with the silanol acidic functional group provided by the hollow fiber itself and also hydrolysis and condensation of silanes of the APS, and the silanol group adjacent to the amine functional group, provide the cooperative acid-base characteristic.

In some embodiments, bromine and fluorine species each individually act as a Lewis acid, so they accept electrons from the donor species. In some embodiments, the proton donors —OH and —NH are each individually electrophiles and act as a Lewis weak acid site (silanol groups) and base site (amine group), respectively.

In some embodiments, the combination of anionic species and cationic species on the body of a single catalyst molecule provides multi cooperative-active sites in a final structure of a multifunctional organocatalyst.

In some embodiments, a chemical reactor comprises the organocatalyst. In some embodiments, a chemical reactor comprises the multifunctional hollow fiber organocatalyst. Reactions can occur in both batch and continuous-flow modes. In some embodiments, a chemical reactor comprises a reactor bed packed with the organocatalyst.

In some embodiments, a process for producing an organocatalyst comprising a porous hollow fiber and at least two functional groups covalently bonded to the porous hollow fiber, wherein the organocatalyst is free of metals, in accordance with the present disclosure comprises (i) spinning a porous hollow fiber, (ii) grafting a first functional group to the porous hollow fiber; and (iii) grafting a second functional group to the porous hollow fiber.

In some embodiments, the porous hollow fiber may be produced according to any suitable method. In some embodiments, the porous hollow fiber is produced according to a spinning method. In some embodiments, the process step of spinning the porous hollow fiber comprises spinning the porous hollow fiber with a technique selected from the group consisting of dry-jet, wet-quench spinning, melt spinning, dry spinning, wet spinning, additive manufacturing (such as 3D printing), and combinations thereof.

In some embodiments, at least one of the process steps of grafting a first functional group to the porous hollow fiber and grafting a second functional group to the porous hollow fiber comprises covalently bonding a functional group to the porous hollow fiber. In some embodiments, grafting a first functional group to the porous hollow fiber and grafting a second functional group to the porous hollow fiber each individually comprises covalently bonding a functional group to the porous hollow fiber.

In some embodiments, a process for reacting chemicals in accordance with the present disclosure comprises (i) introducing a first reactant to an organocatalyst comprising a porous hollow fiber and at least two functional groups covalently bonded to the porous hollow fiber, wherein the organocatalyst is free of metals, (ii) introducing a second reactant to the organocatalyst and (iii) reacting the first reactant and the second reactant.

In some embodiments, the process is a process selected from the group consisting of a continuous flow process, a semi-continuous flow process, a batch flow process, and combinations thereof. In some embodiments, the process is a continuous flow process.

In some embodiments, the process step of introducing the first reactant to the organocatalyst comprises forming a first mixture comprising the first reactant, the organocatalyst, and optionally a solvent.

In some embodiments, the process step of introducing the second reactant to the organocatalyst comprises forming a second mixture comprising the first reactant, the organocatalyst, the second reactant, and optionally a solvent.

In some embodiments, the process step of reacting the first reactant and the second reactant comprises a process step selected from the group consisting of heating the organocatalyst, mixing the second mixture, and combinations thereof. In some embodiments, the process step of reacting the first reactant and the second reactant comprises heating the organocatalyst.

In some embodiments, the process further comprises introducing at least one additional reactant during at least one process step.

In some embodiments, the process step of reacting the first reactant and the second reactant comprises a reaction selected from the group consisting of a heterogeneous reaction, a homogenous reaction, and combinations thereof. In some embodiments, the process step of reacting the first reactant and the second reactant comprises a heterogeneous reaction.

In some embodiments, the first reactant comprises a reactive compound selected from the group consisting of H2O, CO₂, SO₂, SOₓ, NO₂, NOₓ, CO, O₂, CH₄, H₂, N₂O, SH₆, olefins (wherein x=2, 3 . . . ), and combinations thereof. In some embodiments, the first reactant comprises a reactive gas selected from the group consisting of CO₂, SO₂, NO₂, CO, and combinations thereof. In some embodiments, the first reactant comprises a non-reactive compound selected from the group consisting of inert gases, He, Ar, Kr, Xe, N₂, and combinations thereof.

In some embodiments, the second reactant comprises a reactive gas or reactive liquid selected from the group consisting of organic carbonates, alkyl- and aryl sulfides, alkyl- and aryl phosphates, chiral pharmaceutical intermediates, epoxides, aryl- and alkyl amines, and combinations thereof. In some embodiments, the second reactant comprises a reactive gas or reactive liquid selected from the group consisting of organic carbonates, alkyl- and aryl sulfides, alkyl- and aryl phosphates, chiral pharmaceutical intermediates, and combinations thereof. In some embodiments, the second reactant comprises a reactive gas or reactive liquid selected from the group consisting of epoxides, aryl- and alkyl amines, and combinations thereof.

In some embodiments, a method of using an organocatalyst comprising a porous hollow fiber and at least two functional groups covalently bonded to the porous hollow fiber, wherein the organocatalyst is free of metals, comprises using the organocatalyst to catalyze a chemical reaction.

In some embodiments, the chemical reaction is selected from the group consisting of a water purification reaction, a reaction of an organic contaminant in water, a small molecule reaction, a CO₂ reaction, a bioreaction, an enzymatic reaction, and combinations thereof.

The organocatalysts may be used in a variety of applications, including economic and environmentally-sustainable production of pharmaceuticals, fine chemicals, and agrochemicals, water treatment, and upgrading of biomass feedstocks. In some embodiments, the organocatalyst is used in an application selected from the group consisting of water purification, chemical production, biogas and biomass upgrading, and combinations thereof. In some embodiments, the organocatalyst is used to purify water. In some embodiments, the organocatalyst is used to produce valuable chemicals.

### EXAMPLES

Metal- and Solvent-Free Synthesis of Aminoalcohols Under Continuous Flow Conditions.

Examples 1-9 were performed to demonstrate a metal- and solvent-free synthesis of aminoalcohols under continuous flow conditions.

Materials.

The following materials were used herein. 3-aminopropyltrimethoxysilane (APS, 97%), polyvinylpyrrolidone (PVP, average M_{W} of about 1,300,000) and N-methyl-2-pyrrolidone (anhydrous NMP, ACS reagent, >98.5%), dimethylformamide (DMF) (99.8%), acetonitrile (ACN) (99.8%), dimethylformamide (DMF) (99%), dimethylacetamide (DMA) (99.8%), 1,3-dibromopropane (97%), methanol, aniline (99.5%) and propylene oxide (97%) were purchased from Sigma-Aldrich. Polyamide-imide (PAI) was supplied form Solvay Advanced Polymers (Alpharetta, Ga.). Nitrogen was purchased from Airgas and used to purge the sample in the continuous flow system.

### Example 1. Polymer Dope Composition and Creation of Polyamide-Imide Hollow Fibers

A porous polyamide-imide hollow fiber (PAI) was synthesised according to a conventional dry-jet, wet-quench spinning method. Torlon 4000T-HV, a commercially available polyamide-imide PAI and PVP were used for the synthesis of the composite hollow fiber catalysts. The PVP was dried at 80° C. for 24 hours under vacuum to release pre-sorbed water vapor. After that, Torlon was dried at 110° C. for 24 hours proceeding using De-ionized (DI) water that was added as a nonsolvent into the fiber dope. NMP was embedded as the solvent to form the spinning dope, which could attribute to its strong solvent power, low volatility, and good water miscibility. All solvents and nonsolvents were used as-received with no purification or modification. Methanol and hexane were used for solvent exchange after fiber catalyst spinning. Methanol was also used to remove excess water from the fibers. Spinning dope to create a 10/90 (weight ratio) catalyst/Torlon contains the polymer PAI, NMP (solvent), water (nonsolvent), and additives (PVP). The optimized polymer dope compositions and spinning conditions are tabulated in Table 1.

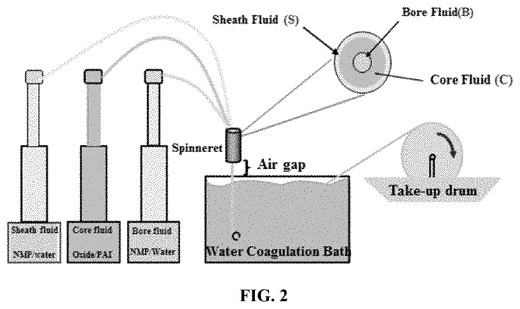

FIG. 2 shows a schematic representation of the hollow fiber creation process. A standard bore fluid involved in the present disclosure consists of NMP and water with the weight ratio of 88:12. An appropriate Torlon core dope composition (determined by the cloud point method and rheology measurements) was fed to the middle spinneret compartment.

TABLE 1
Optimized spinning conditions for corresponding of polyamide-imide
hollow fibers.

| Parameter | Optimized Value |
|---|---|
| Dope composition | 24/7/64.5/4.5 wt % |
| (PAPPVP/NMP/H₂O) | |
| Dope flow rate | 600 mL/hr |
| Shear fluid (NMP/H₂O) | 50/50 wt % |
| Shear flow rate | 50 mL/hr |
| Bore fluid (NMP/H₂O) | 88/12 wt % |
| Bore fluid flow rate | 200 mL/hr |
| Air gap | 10 cm |
| Take up rate | 8.5 m/min |
| Operating temperature | 60° C. |
| Quench bath temperature | 60° C. |

### Example 2. Post Spinning Infusion and Amine Grafting of Polyamide-Imide Hollow Fibers

APS was used as the grafting agent for grafting zirconia-Torlon fiber catalysts. The amine grafting was performed in a mixture of a non-polar solvent (toluene) and a polar protic solvent (water). The water content of the mixture was kept within the range between about 0.01-1.00 wt %. Water has played a vital role for protonating APS and hydrolyzing methoxy groups in dry liquid. Exposure of APS to moisture obstructs strong hydrogen bonds leading to the formation of polysiloxane. A water content of 1.00 wt % caused the rise to highest amine loading. As described above, after the PAIHFs composites were formed, they were subjected to a methanol solvent exchange process demonstrated by exposure to different APS/toluene/water (ratio 9.9:90.0:0.1 wt %) solution mixtures with varying immersion times, from 1 to 6 hours. As a result, the PAIHFs catalysts were removed from the amine solution and rinsed with hexane for 30 minutes at ambient temperature to wash away the ungrafted APS deposited onto the fiber surface. APS-grafted PAIHF catalysts were placed in a preheated vacuum oven and cured for 1 hour at 60° C. APS aminosilane can form a durable bond with the PAIHFs in this post-spinning immersion step. The optimum infusion condition was determined by soaking 0.15 g of PAIHF catalysts in a 100 g solution of different concentrations of APS in toluene/water mixture (i.e., 5, 10, 15, and 20 wt % APS) at room temperature for 1-8 hours. The fibers exhibited an optimum amount of amine loading for 10 wt % APS/toluene/water solution and an infusion time of 2 hours.

The APS grafted PAIHFs were further modified by reaction with 1,3-dibromopropane in dry toluene at 80° C. for 2 hours to yield the ammonium bromide derivatives. Finally, the fibers were solvent exchanged with toluene and dried under vacuum (30 mTorr) at 85° C. to remove the residual solvent from the pores, yielding the Br-immobilized APS-grafted PAIHFs (Br/APS/PAIHFs). The synthesis reaction is shown in (Scheme 1).

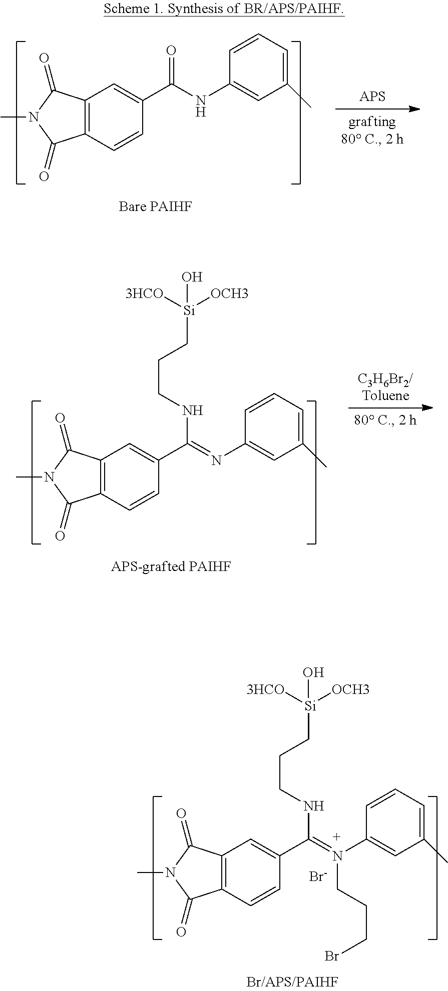

### Example 3. Characterization of Br/APS/PAIHF Trifunctional Organocatalysts and Reaction Products

The Br/APS/PAIHF structure of Example 2 was confirmed by Fourier transform infrared spectroscopy (FTIR) in the range of 400-4000 cm⁻¹ by a Bruker Tenser instrument. The spectra were acquired at a 4 cm⁻¹ resolution. CHN analyses (PerkinElmer Series II, 2400) and inductively coupled plasma optical emission spectroscopy (ICP-MS) were used to determine amine and bromine loadings of the hollow fibers. The BET surface area, BJH pore volume, and average pore size of bare PAIHF, APS/PAIHF and Br/APS/PAIHF were determined using nitrogen isotherms (−196° C.) in a Micromeritics 3Flex. Prior to analysis, the hollow fibers were degassed at 110° C. under vacuum for 12 hours to remove any pre-adsorbed species from the pores of the materials. X-ray photoelectron spectroscopy (XPS) was carried out to map the presence of various elements in the hollow fiber surface. The CO₂ adsorption capacity of the fibers were measured at 35° C. in 10% CO₂ concentration (balanced with N2) using TGA. The corresponding results are tabulated in Table 2. The high-resolution scanning electron microscope (Hitachi S-4700 FE-SEM) was used to assess the morphology of the bare and grafted Br/APS/PAIHFs. The product was analyzed on a 7890B Agilent Gas Chromatograph (GC) equipped with a flame ionization detector (FID) for analyzing propylene oxide, propylene carbonate, aniline, and amino alcohols. A DB WAX column (30 m×0.320 mm×0.25 μm) was used for separation. In addition, the analysis of products was performed by using ¹H nuclear magnetic resonance (NMR) and ¹³C NMR spectra at room temperature using a Bruker-DRX 400 MHz spectrometer ¹H liquid-state NMRCPMAS TOSS (¹H NMR).

### Example 4. Tandem Batch Reaction of CO₂ and Propylene Oxide with Aniline

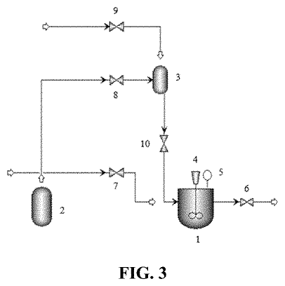

To examine the performance of Br/APS/PAIHF catalyst, a two stage tandem reaction was carried out in a high-pressure laboratory autoclave (Parr, USA) that was equipped with a stirrer and jacket heater. The experimental setup is shown in FIG. 3. The numbers represent elements as follows: reactor 1; gas cylinder 2; aniline cylinder 3; stirrer 4; pressure gauge 5; needle valves 6, 7, 10; ball valves 8, 9. In a typical catalytic reaction, 2 mL (30 mmol) of propylene oxide, 30 mL of DMA (as a solvent), and 50 mg of Br/APS/PAIHF catalyst were delivered into a high-pressure laboratory autoclave. After being sealed, the reactor was carefully flushed three times with CO₂. After flushing, 5-50 bar of CO₂ was pressurized in the reactor and heated up to 140° C. under stirring for 1-6 hours. A number of samples were collected during the reaction and analyzed by GC-MS. Then, the required amount of aniline (e.g., 2.7 ml, 30 mmol) was introduced for further reaction with obtained intermediate propylene carbonate for 1-6 hours at 140° C. (Scheme 2). After the reaction was completed, the autoclave continued to cool down at room temperature, depressurized, and opened slowly. The reaction mixture was separated by filtration (Whatman, 0.22 μm) then analyzed by GC-MS and GC-FID. As a control material, bare PAIHF and APS/PAIHF were also evaluated at the same conditions.

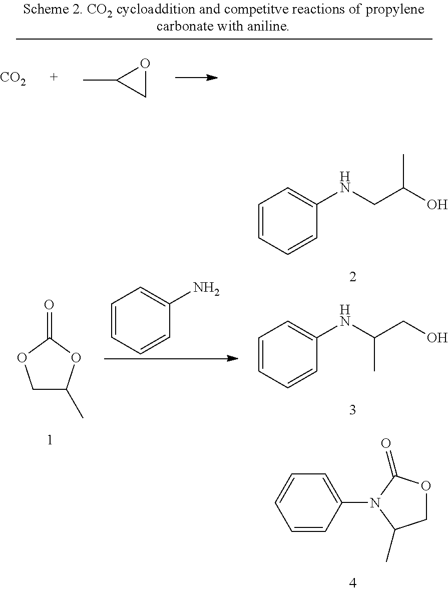

### Example 5. Batch Reaction of Propylene Carbonate with Aniline

Batch reaction of propylene carbonate with aniline was performed in the presence of bare PAIHF, APS/PAIHF and Br/APS/PAIHF in a 25 mL two-neck flask reactor equipped with a reflux glass condenser. Both propylene carbonate and aniline are liquids and not flammable; therefore, they can be used as stoichiometric reagents without added solvents. In a typical procedure, a 10 mL mixture of propylene carbonate and 10 mL aniline were added to a clean flask followed by the addition of about 100 mg of hollow fiber catalysts into the solution. The system was then heated under a thermostatically and timer-controlled oil bath with a magnetic stir bar. The experiments over various hollow fiber catalysts were conducted at 140° C. and different reaction times (1-48 hours) to identify the best reaction conditions that yield the best reaction performance. Samples were taken and filtered every hour for analysis in GC-MS where methanol was used as solvent.

### Example 6. Continuous Reaction of Propylene Carbonate with Aniline

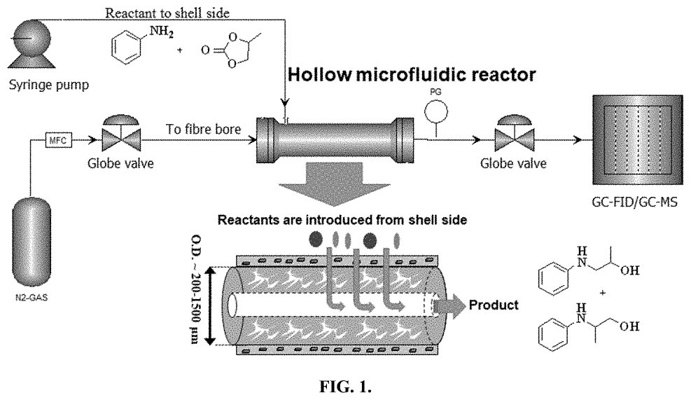

To test the metal-free Br/APS/PAIHF trifunctional organocatalyst as a heterogeneous catalyst and continuous-flow reactor, a hollow fiber module containing five fibers (with an inner diameter of 0.1 μm, length of 25 cm, and total volume of 15 mL) was formed and the catalytic reaction between propylene carbonate and aniline was performed. The reactor was made of a stainless-steel tube with an inner diameter of 6.3 mm. In a typical experiment, self-supported fibers (e.g. 3-5 fibers) were packed in the hollow fiber module. To start the reaction, the solution of mixtures (propylene carbonate:aniline, 1:1 ratio) was introduced to the hollow fiber shell side at a flow rate of 0.02-0.08 cm³/min (0.02 cm³/min is equivalent to 10 mL of propylene carbonate and 10 mL aniline with 100 mg hollow fiber catalysts in batch system) at 140° C., while nitrogen was introduced to the hollow fiber bore side at 15 mL/min flow rate to prevent pore blockage as well as to push the product out of the bore. The schematic diagram of the experimental set-up is shown in FIG. 1. The reactor was left for half an hour to reach steady state, then samples were collected every 30 min.

### Example 7. Br/APS/PAIHF Trifunctional Organocatalyst Characterization

As described herein, species were bonded covalently in the highly interconnected pore cell walls of the polymer surface for an effective catalytic and continuous flow reaction. Hence, a combination of the fiber's textural properties and catalytic test results provides useful information to gain insight into the catalytic immobilization of trifunctional organocatalysts and their performance in continuous flow processes. The textural properties and elemental analysis of bare PAIHF, APS/PAIHF and Br/APS/PAIHF before and after reaction were characterized, and the corresponding results are summarized in Table 2. Both the surface area and pore volume of hollow fibers were reduced by approximately 50% after fiber treatment with APS and bromine solutions respectively, which could be due to polymer chain rearrangement and successful grafting of APS and bromine into bare PAIHF. These results show that the post-treatment of fibers may swell the polymer chains thereby resulting in a smaller pore size towards the surface. The textural characteristics of Br/APS/PAIHFs were also investigated and showed very similar characteristics before and after catalytic evaluation.

TABLE 2
Textural properties, CO₂ capacity, amine, and bromide loading of the
bare PAIHF, APS/PAIHF and Br/APS/PAIHF before and after reaction.

| | | | Br/APS/ | Br/APS/ |
|---|---|---|---|---|
| | Bare | APS/ | PAIHF | PAIHF |
| Hollow fibers | PAIHF | PAIHF | (before reaction) | (after reaction) |
| SBET (m²g⁻¹) | 57.54 | 25.20 | 15.60 | 14.05 |
| Vpore (cm³g⁻¹) | 0.27 | 0.17 | 0.10 | 0.10 |
| CO₂ capacity | 0.03 | 1.31 | 1.42 | 1.37 |
| (mmol g⁻¹ | | | | |
| fiber) | | | | |
| N loading | — | 3.90 | 3.70 | 3.70 |
| (mmol g⁻¹ | | | | |
| fiber) | | | | |
| Br loading | — | — | 0.10 | 0.10 |
| (mmol g⁻¹ | | | | |
| fiber) | | | | |

^{a}Determined by nitrogen physisorption experiments at 77K.
^{b}Determined by BJH method.
^{c}Measured by TGA at 35° C. in 10% CO₂ concentration (balanced with N₂).
^{d}Determined by elemental analysis.

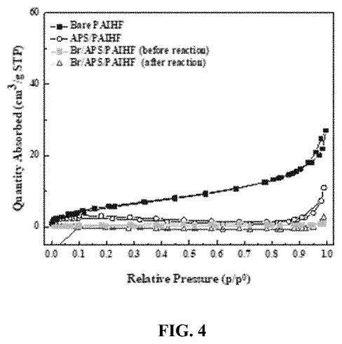

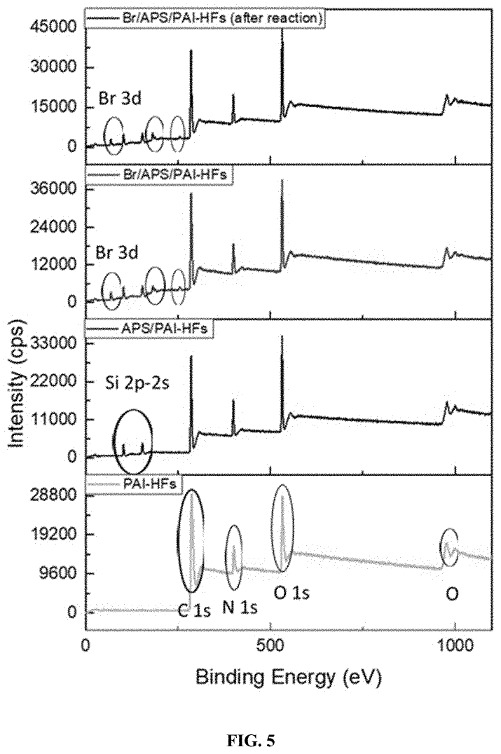

The N₂ physisorption isotherms (FIG. 4) of bare PAIHF, APS/PAIHF and Br/APS/PAIHF before and after reaction are type-IV isotherms with H₁ type hysteresis which indicates a typical mesoporous structure according to IUPAC classification. Furthermore, it was observed that the CO₂ capacity uptake over fiber increases with APS and bromide grafting, which is important for the CO₂-propylene oxide cycloaddition reaction step. Moreover, the successful loading of APS and bromine in the PAIHF was confirmed by the ICP (Table 2) and XPS analyses (FIG. 5). The results of XPS indicate negligible changes in the chemical nature of PAIHF after grafting with APS and bromine. Qualitative elemental analysis revealed that nitrogen and bromine loading in Br/APS/PAIHF were similar before and after reaction, implying that the chemical state of fibers remained unchanged after reaction. The loading of nitrogen and bromine to 1 g of fiber were around 3.7 and 0.1 mmol, respectively.

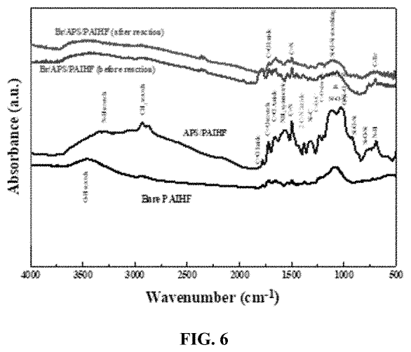

The FTIR spectra of all hollow fibers are shown in FIG. 6. The spectra at 730 cm⁻¹ and 1619 cm⁻¹ were ascribed to the presence of the Si—O bond and conjugated C=N vibration of the cyclic system (imidazole), respectively. The vibration band at 597 cm⁻¹ demonstrated the functionalization of hollow fibers with bromide. The post-treated fibers show a broad vibration band in the range of 3000-3500 cm⁻¹ and 3468-3416 cm⁻¹ attributed to the O—H hydrogen bonds in SiO—H (from APS grafting) and the N—H (from APS grafting and polymer backbone) vibration bands, respectively. The IR spectrum of the Br/APS/PAIHF confirmed the successful aminosilane and bromide grafting on the surface of PAIHF.

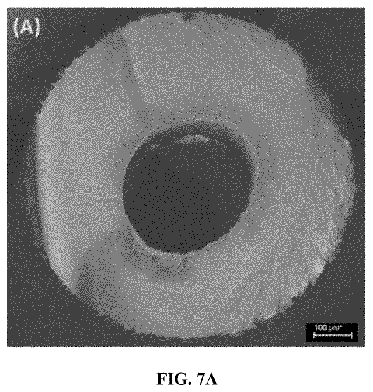

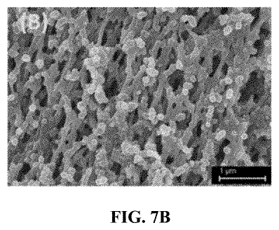

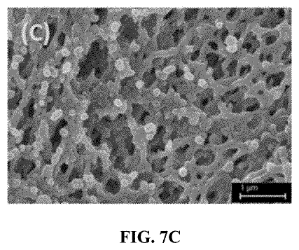

FIG. 7 shows SEM images of the cross-section (a) and surface of Br/APS/PAIHF before (b) and after (c) catalytic reaction. The fibers were rinsed with hexane after each post-treatment and catalytic reaction to prevent substructure collapse and maintain the permeability of the hollow fiber microfluidic reactors. The micrographs qualitatively confirm that the fiber pore morphology slightly collapsed after catalytic processing which is in agreement with the fibers textural properties. However, the surface layer still remained uniform and showed moderate porosity after multiple exposures to various solvents (FIG. 7C) which confirms a robust structure of PAI polymer matrix.

### Example 8. Initial Assessment of the Catalytic Activity of Br/APS/PAIHF Trifunctional Organocatalysts in Batch Reaction

The cycloaddition of CO₂ to propylene oxide (PO) for propylene carbonate (PC) formation, which is an intermediate for the further hydroxyalkylation of aniline, was conducted in DMA to evaluate Br/APS/PAIHF trifunctional organocatalysts catalytic activities. The use of the activating abilities of the catalytic medium and the reaction conditions for cycloaddition of CO₂ to epoxide played vital roles in the synthesis of cyclic carbonate. For the present trifunctional organocatalysts, their structures consist of hydrogen-bond donor groups (—OH and —NH), and nucleophilic [Br⁻] groups. These multiple active sites on Br/APS/PAIHF organocatalysts can cooperate to promote CO₂ capture and activation and the ring-opening of propylene oxide substrate. The control experiments were performed with bare PAIHF and APS-grafted PAI-HF. However, no product was detected over these materials. It should be noted that the APS-grafted PAI-HF showed high bulk strength and prevented fiber swelling compared to bare PAIHF that dissolved in DMA. The amine and hydroxyl functional groups in APS grafted hollow fibers are also utilized as hydrogen-bond donor groups for cooperative catalytic reactions.

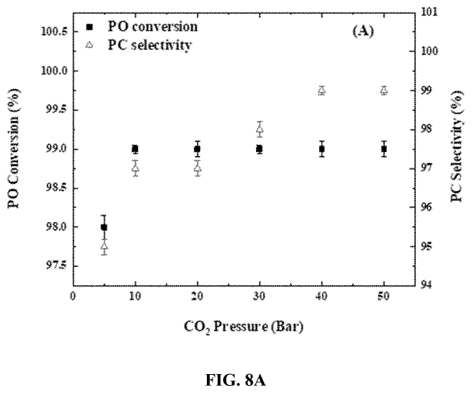

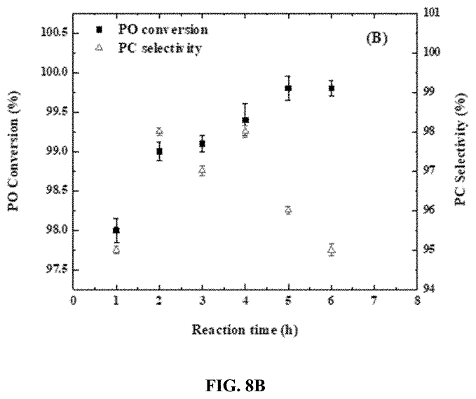

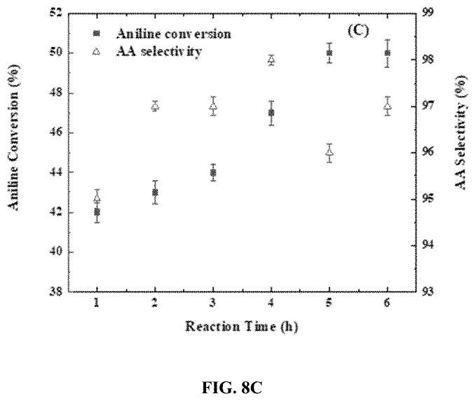

Initially, the influence of the CO₂ pressure on the cycloaddition of CO₂ was evaluated (FIGS. 8A-8C). As shown in FIG. 8A, when CO₂ pressure was increased from 5 to 20 bar, the PC yield was enhanced from 93% to 97%. The PO conversion was smoothly improved to 100% due to the favorable contact between CO₂ molecules and the substrate (PO) phase. When the CO₂ pressure was further increased, the PC selectivity improved with no conversion loss. Therefore, it was found a CO₂ pressure of 20 bar is sufficient for the CO₂ cycloaddition reaction.

FIG. 8B shows the kinetic plot for PC synthesis catalyzed by Br/APS/PAIHF. The PO conversion and PC selectivity were both increased with longer reaction times. When the reaction time was further prolonged, the CO₂ cycloaddition of PO gave rise to product mixture of PC and some aldehyde was obtained, which is consistent with previous reports. Hence, a reaction time of 1 hour was sufficient to obtained 98% PO conversion and 95% PC selectivity.

Immobilized organocatalyst polymeric hollow fibers were cut into pieces and used as heterogeneous catalysts in batch reactor for the tandem reaction of CO₂ with PO and hydroxyalkylation of aniline with obtained intermediate PC at 5 bar CO₂ pressure and 140° C. The results are demonstrated in FIG. 8C. As shown, initially the 1-(phenylamino)propan-2-ol (AA) selectivity was significantly enhanced to 98% overtime (4 hours), and slightly dropped (96%) with further reaction time. However, the aniline conversion reached 98% after 5 hours reaction, and remained constant at longer reaction time.

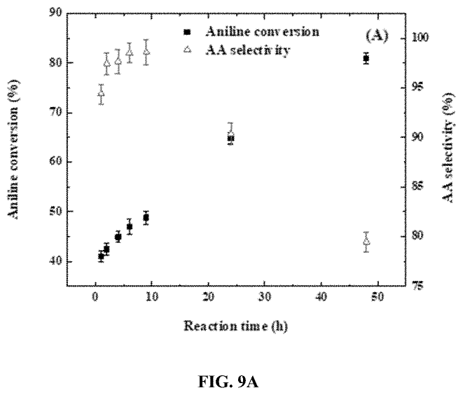

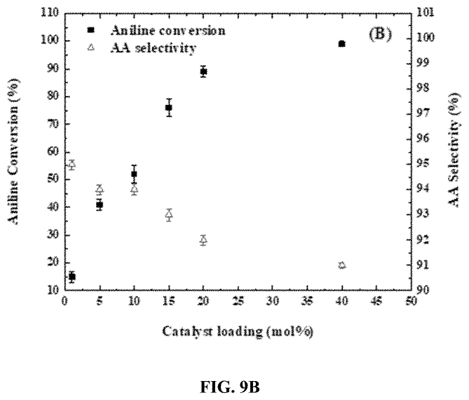

Two sets of experiments were carried out (i) at different reaction times, and (ii) various catalyst loading. In the first set, a mixture of aniline and propylene carbonate (same molar ratio) was set to react at 140° C. in the presence of the same amount of Br/APS/PAIHF catalyst (5 mol % toward to aniline amount). The reactions were monitored by GC-MS at time intervals of 1-48 hours (FIGS. 9A-9B). The reactions of FIGS. 9A-9B used a 10 mL mixture of propylene carbonate and 10 mL aniline and 100 mg of hollow fiber catalysts.

As shown in FIG. 9A, the aniline conversion increased with time, but, the 1-(phenylamino)propan-2-ol selectivity dropped from about 95% to 80%. Another approach to improve the catalytic activity and selectivity is to increase the density of bifunctional species for cooperative interaction. Therefore, a second set of reactions containing an equimolar mixture aniline and PC was set to react with increasing amounts of catalyst and sample at the same reaction time. Initially, the aniline conversion was significantly improved with increasing Br/APS/PAIHF loading of 5 to 20 mol % (toward to aniline amount). When the catalyst loading was further increased, it did not markedly promote the reaction, as seen by a significant drop in 1-(phenylamino)propan-2-ol selectivity after 10% catalyst loading (FIG. 9B).

Without being bound to any particular theory, this could be due to competitive acylation followed by cyclization (Scheme 3, path a). For both sets, the major product was the 1-(phenylamino)propan-2-ol (Scheme 3, compound 2). In the presence of different catalysts, the efficiency of the reaction may be limited by the dual electrophilic character of the propylene carbonates which may cause competitive acylation followed by cyclization (Scheme 3, path a) or hydroxyalkylation (Scheme 3, path b). The results herein clearly show that 1-(phenylamino)propan-2-ol (Scheme 3, compound 2) and 2-anilino-1-propanol (Scheme 3, compound 3) are the major compounds at shorter reaction time, while 3-phenyloxazolidin-2-one (Scheme 3, compound 4) will be more dominant at longer reaction time. It still remains highly challenging and attractive to selectively prepare aminoalcohols through a site-specific hydroxyalkylation reaction using aromatic amines and cyclic carbonates under mild reaction conditions. Although the corresponding knowledge of the acid-base-nucleophilic trifunctional cooperativity is still limited, this new and sustainable catalytic process would represent a valuable alternative to metal-based processes.

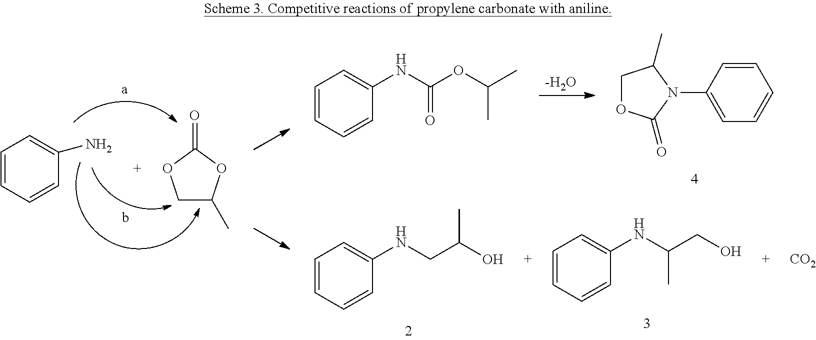

### Example 9. Catalytic Activity in Continuous Microfluidic Reactor

The novel hollow fiber catalyst morphologies and structures described herein offer advantages over fixed-bed and multistep continuous flow reactors by providing a high surface area to volume ratio, avoiding particle attrition and quenching operation, and the providing the ability to minimize mass transfer resistances. In particular, as a result of tunable surface porosity in hollow fiber structures and trifunctionalized organocatalysts, the reaction capabilities of certain composite hollow fiber catalysts are shown herein to meet or exceed those achievable by ionic liquids or other trifunctional catalysts in batch.

The present disclosure defines the theoretical and practical fundamentals of permanent immobilization of Br/APS/PAIHF trifunctional organocatalysts for continuous-flow synthesis of aminoalcohols. After demonstrating the efficiency of Br/APS/PAIHF in hydroxyalkylation of aniline with cyclic carbonate in the batch reactor, the performance of the catalyst was further evaluated over metal-free Br/APS/PAIHF hollow fiber as a heterogeneous catalyst and continuous-flow reactor. The hollow fiber module was formed using five Br/APS/PAIHF fibers. The volume of reactor can vary, depending on the internal diameter of the tubing and number and diameter of hollow fibers used. The performance of the Br/APS/PAIHF module under continuous flow reaction was investigated at different flow rates (0.02-0.08 cm³/min) at 140° C. for 6 hours while samples were collected for analyses every 30 minutes. The reaction was performed under the same conditions as the packed-bed reaction, but the reactant traveled from the shell side through the porous hollow fiber catalyst, then product out into the bore.

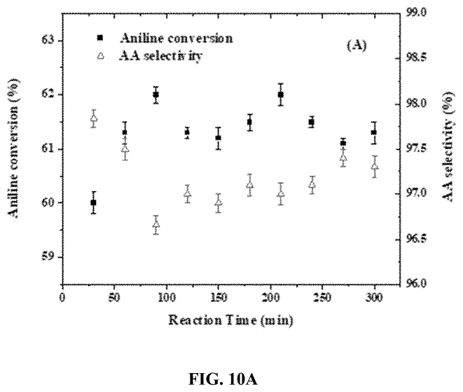

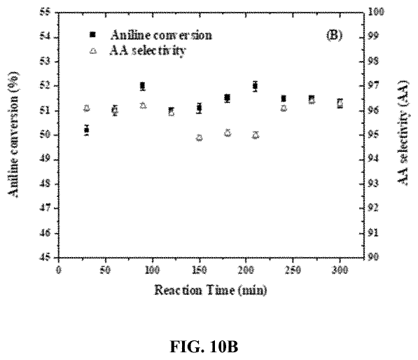

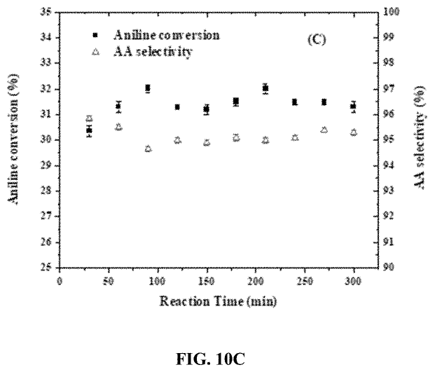

A syringe pump was used to continuously introduce reactants at 0.02, 0.04 and 0.08 cm³/min to the shell side of the hollow fiber microfluidic reactor at 140° C. The relationship between catalyst performance and flow rate in continuous hollow fiber module for hydroxyalkylation of aniline with cyclic carbonate are shown in FIGS. 10A-10C. The structure and the purity of the products were identified by GCMS, ¹H NMR and ¹³C NMR.

The highest conversion value of 61% and selectivity value of 97.5% towards 1-(phenylamino)propan-2-ol was achieved at the lowest flow rate (0.02 cm³/min) whereas, by increasing flow rate to 0.08 cm³/min, conversion dropped drastically to 30% while selectivity remained stable (about 95%) for the hydroxyalkylation of aniline. The same hollow fiber module was reused at each flow rate after rinsing with hexane then drying under a vacuum at 80° C. for 2 hours without obvious loss of activity. The results show the synergistic cooperative effect of trifunctional organocatalysts on PAIHF.

In the present disclosure, the organocatalysts were immobilized onto and into the sponge wall or shell-side of the hollow fiber; hence, the reactant molecules need more time to diffuse into the pores and come into contact with the catalytically active sites compared to homogeneous reaction system. Therefore, it is reasonable that, in a heterogeneous hollow fiber reaction system, substrate concentration and volume may lead to improved mass transport performance and enhanced reaction kinetics. Since the same reaction mixture was used, the reaction rate difference may be due to the high availability of active sites on hollow fiber catalysts and removing the products from reaction media. The product and reaction mixtures were removed from the hollow fiber module continuously, and the conversion of aniline reached 61% after 30 min reaction under steady state condition (at a flow rate of 0.02 cm³ min⁻¹, residence time of about 5 min). The performance enhancement is normally attributed to larger surface area and a number of surface atoms, leading to more active sites. Also, as evident from FIG. 10A, the catalyst retained its activity after 300 min. These findings demonstrate that the trifunctional organocatalyst microfluidic reactor described herein yields almost the same conversion and selectivity as batch while being resistant to deactivation.

It is also worth noting that Br/APS/PAIHF trifunctional organocatalysts were more selective toward 1-(Phenylamino)propan-2-ol (Scheme 3, compound 2) products than the 3-phenyloxazolidin-2-one product (Scheme 3, compound 4) compared to those for batch reaction. This finding further confirms that the Br/APS/PAIHF continuous flow microfluidic reactor is not only an efficient system to perform ultrafast exothermic reactions, but also offers a unique way to control the reactions which proceed via highly unstable intermediates.

Contrary to batch reaction where the majority of products were 3-phenyloxazolidin-2-one (Scheme 3, compound 4), continuous flow reactor gave exclusively (or in a major proportion) the aminoalcohol product. The implications of the present disclosure include the possible synthesis of an important chemical precursors (the 1-(Phenylamino)propan-2-ol) in pure form and in high yield.

The resulting self-supported catalysts are stable and well-behaved under catalytic conditions and demonstrate outstanding reactivity and selectivity, comparable to or exceeding their analogous homogeneous counterparts. Incorporating trifunctional organocatalysts in the walls of the fibers presents an alternative to a traditional packed-bed reactor. A continuous flow reaction system is an ideal platform for the hydroxyalkylation of aniline because the reaction proceeds through proton transfer between reactants, thereby affording the desired products by simply controlling contact time and reaction temperature.

The results of these experiments show high efficiency of the catalyst and very short reaction time compared to the other reactions, and the data show that after only 30 minutes of reaction time a conversion of 30% was achieved with 97% selectivity and 29% yield which indicates a much higher efficiency for this process. The reaction was left to run for 6 hours and the end result was 60% conversion, 97% selectivity, and 59% yield. Without being bound to any particular theory, one reason for these results could be the structure of the hollow fiber and the process of traveling from the shell side through pores to the hollow side and exiting maximizes the contact area with the active sites on the catalyst. Another reason could be the lack of air in the process which is present in the batch reactor continuous microreactor setup ensures that air cannot get into the process.

As a sustainable alternative for conventional batch-based synthetic techniques, the concept of hollow fiber catalysts for continuous-flow processing has emerged in the synthesis of fine chemicals. The fiber synthesis allows for a variety of supported organocatalysts to be immobilized in the fiber pores, thus leading to a diverse set of reactions that can be catalyzed in flow. Additionally, the fiber synthesis is a scalable, versatile, and user-friendly system wherein only reactor modules would be attached by the operator in a matter of seconds, without further reconfiguration of pumps, tubes, and other flow components that makes it particularly attractive for any type of reactions. Chemically compatible continuous flow reaction components enable various chemical transformations on a small footprint platform providing superior mixing for immiscible reagents, efficient heat transfer and having an intuitive graphical user interface with a practical engineering solution.

This type of catalyst is a very innovative system that can be utilized in various applications. The results show that the conversion and selectivity are the highest in the pressurized batch system, but when it is compared to the continuous flow reactor, higher numbers of conversion, yield, and selectivity can be achieved in a shorter time than the batch system. The structure and properties of the hollow fiber play a significant role in the reaction providing a large surface area compared to volume which is excellent for the mass transfer process and low flow resistance in the continuous flow reactor. This reconfigurable system has accelerated the synthesis of lab-scale quantities of organic precursors. Further, the small footprint and user-friendly nature of the developed system makes it a particularly attractive system to optimize and evaluate various chemical transformations in a matter of hours or days and do so under identical reaction conditions and allows researchers to direct more of their efforts toward the creative aspects of process intensification. The present disclosure provides a new prospect for permanent immobilization of multifunctional organocatalysts that is simple, fast, and promising for selective and sustainable chemical transformation.

Optimized Immobilization Strategy for Trifunctional Organocatalysts for Synthesizing Amino Alcohols Under Mild Reaction Conditions.

Examples 10-24 were performed to optimize the immobilizing strategy for synthesizing amino alcohols under mild reaction conditions.

Materials.

The following materials were used herein. Sodium 2-Bromoethanesulfonate, 3-Bromopropanesulfonic, Nonafluorobutane-1-sulfonic acid, propylene carbonate (PC), aniline, hexyl amine, butyl amine, 4-aminophenol, toluene, 3-aminopropyltrimethoxysilane (97%) (APS), and methanol were purchased from Sigma-Aldrich; all the chemicals were used as received without more purification. Polyamide-imide (PAI) was supplied form Solvay Advanced Polymers (Alpharetta, Ga.). Nitrogen was purchased from Airgas and used to purge the sample in the continuous flow system.

### Example 10. Formation of Hollow Fiber Polyamide-Imide (HF-PAI)

Composite hollow fibers (HFs) were synthesized from commercially available polyamide-imide (PAI), which had the property of resisting heat and swelling, via the template-free, dry-jet, wet-quench spinning method. HF-PAI had a high porosity and surface area; this property made them available for functionalization by the various organic species. This hollow fiber material has been applied in various gas separation processes successfully, such as working excellently in the CO₂ capture, but having been worked as catalysts in the organic reactions.

This HF membrane reactor was the first instance of a HF membrane reactor being investigated and developed into the laboratory-scale continuous-flow reactor system for various sustainable chemical formation reactions. The stability, and compatibility of HF-PAI, were improved by soaking them in commonly used solvents, such as DMA and DMF. HF-PAI was subsequently treated and bonded with a mixture of toluene, APS, and water in a weight ratio of 90:9:0.1 respectively at 80° C. for 2 hours to obtain HF-PAI/APS.

### Example 11. Synthesis of Multifunctional Organocatalysts [HF PAI/APS/3-Br-Prop]

In a 100 mL round bottom flask, 40 ml of methanol (1.019 mole) was added to 3-bromopropanesulfonic (1 g) and was stirred until the 3-bromopropanesulfonic completely dissolved in methanol. Then HF-PAI/APS (2 g) was introduced to the mixture, which was stirred under a reflux condenser for 6-8 h at 80° C. in an oil bath to give the ammonium bromide derivatives. Finally, the fibers were solvent exchanged with toluene and dried under a vacuum (30 mTorr) at 80° C. to remove the residual solvent from the pores, yielding the Br⁻ immobilized APS-grafted PAIHFs, [HF-PAI/APS/3-Br-Prop]. The synthesis reaction is shown in Scheme 4.

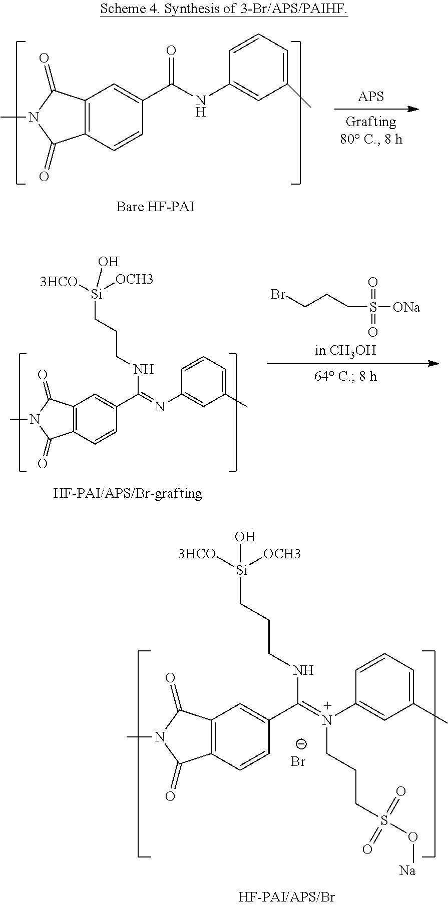

### Example 12. Synthesis of Multifunctional Organocatalysts [HF PAI/APS/Na-2-Br]

Synthesis of multifunctional organocatalysts [HF-PAI/APS/Na-2-Br] Sodium 2-Bromoethanesulfonate (1 g) was completely dissolved in 40 mL methanol (1.019 mole), the mixture was transferred to a 100 mL round bottom flask, then (2 g) of HF-PAI/APS was carefully added to the mixture. The obtained mixture was stirred under a reflux condenser at the same conditions mentioned above, and the immobilized result was washed by toluene to remove the remaining methanol. It was dried under a vacuum (30 m Torr) at 80° C. to remove the residual solvent from the pores, yielding the Br-immobilized APS-grafted PAIHFs [HF-PAI/APS/Na-2-Br. The synthesis reaction is shown in Scheme 5.

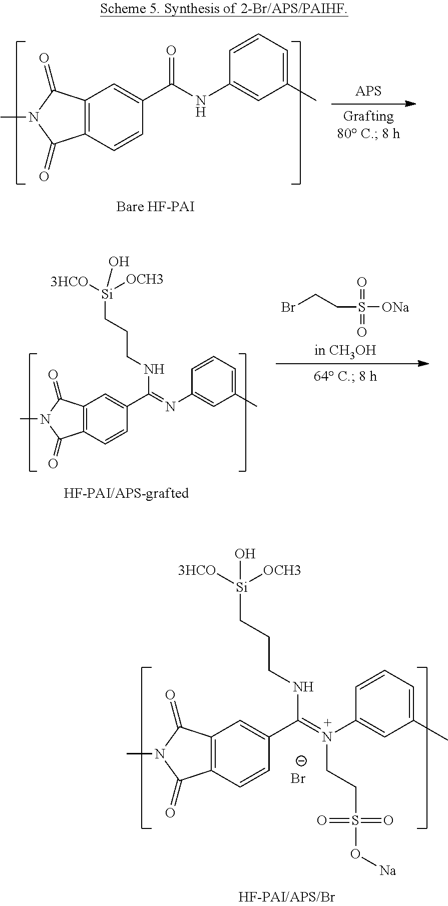

### Example 13. Synthesis of Multifunctional Organocatalysts [HF-PAI/APS/F]

Nonafluorobutane-1-sulfonic acid (1 g) was completely dissolved in 40 mL methanol (1.019 mol), the mixture was transferred to a 100 mL round bottom flask, then 2 grams of HF-PAI/APS was carefully added to the mixture. The combined mixture was stirred under a reflux condenser at the same conditions mentioned in the above example, the resulting product was washed by toluene to remove the remaining methanol, and then it was dried under a vacuum at 80° C. to remove the residual solvent from the pores. This process yielded the F⁻ immobilized APS-grafted PAIHFs [HF-PAI/APS/(Nonafluorobutane-1-sulfonic acid)]. The synthesis reaction is shown in Scheme 6.

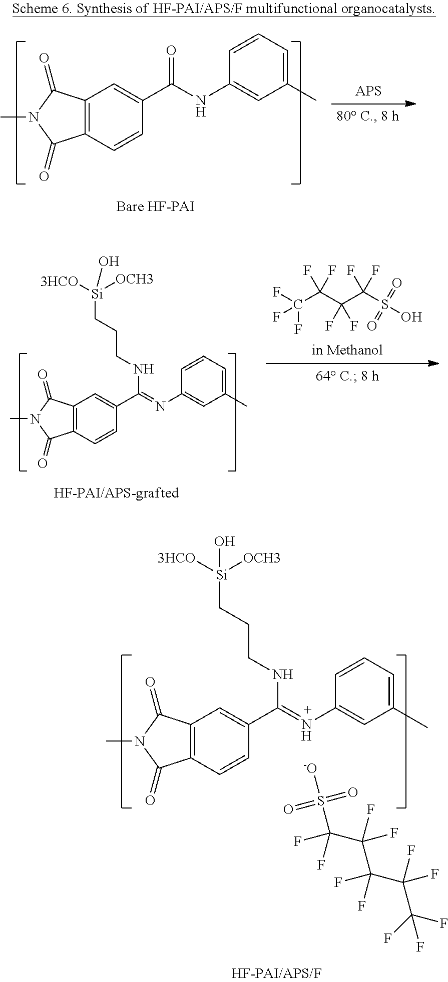

### Example 14. Characterization of Multifunctional Organocatalysts

The structure of multifunctional organocatalysts [HF-PAI/APS/3-Br-Prop], [HFs-PAI/APS/Na-2-Br], and [HF-PAI/APS/F] were demonstrated by Fourier Transform Infrared Spectroscopy (FTIR) in the range of 400-4000 cm⁻¹ with a Bruker Tensor instrument. The spectra were acquired at a 4 cm⁻¹ resolution. The BET surface area, BJH pore volume, and average pore size of bare [HFs-PAI,] [HFs-PAI/AP S], [HFs-PAI/APS/3-Br-Prop], [HFs-PAI/APS/Na-2-Br], and [HF-PAI/APS/F] were determined using nitrogen isotherms (−196° C.) with a Micromeritics 3Flex. Prior to analysis, the hollow fibers were degassed at 110° C. under vacuum for 2 hours to remove any pre-adsorbed species from the pores of the materials' pores. The CO₂ adsorption capacities of the fibers were measured at 35° C. in a 10% CO₂ atmosphere (balanced with N₂) using TGA. The characterizations results are shown in Table 3.

Inductively coupled plasma optical emission spectroscopy (ICP-OES) was used to determine the amine and bromine loadings of the hollow fibers. The corresponding results are given in Table 3. A high-resolution scanning electron microscope (Hitachi S-4700 FE-SEM) was used to assess the morphology of the bare HFs-PAI and I-ifs-PAI/APS. The products and reactants were analyzed on an Agilent 19091S-4 33 gas chromatograph (GC) equipped with a mass spectroscopy (MS). The method and capillary column mode are as follows, HP-5MS (0.25 mm×30 m×0.25 μm); Max temperature: 350° C. Nominal length: 30.0 m, Nominal diameter: 250.00 μm, Nominal film thickness 0.25 μm; Mode constant pressure 14.70 psi; Nominal initial flow 1.8 mL/min Average velocity: 49 cm/sec. Beyond GC-MS, the products were determined by using ¹H nuclear magnetic resonance (NMR) and ¹³C NMR at room temperature using a Bruker-DRX 400 MHz spectrometer.

TABLE 3
Textural properties, CO₂ capacity, amine and bromide loading of the
bare [HF-PAI], [HF-PAI/APS], [HF-PAI/APS/Na-2-Br], [HF-PAI/APS/
3-Br-Prop], and [HF-PAI/APS/F].

| | | HF- | HF- | HF- | HF- |
|---|---|---|---|---|---|
| | HF- | PAI/ | PAI/APS/ | PAI/APS/3- | PAI/ |
| Hollow fibers | PAI | APS | Na-2-Br | Br-Prop | APS/F |
| SBET (m²/g)^{a} | 57.54 | 25.20 | 16 | 16.5 | 17 |
| V pore (cm³ g⁻¹)^{b} | 0.27 | 0.17 | 0.119 | 0.118 | 0.092 |
| CO₂ capacity | 0.039 | 1.32 | 1.42 | 1.5 | 1.4 |
| (mmol per g | | | | | |
| fiber)^{c} | | | | | |
| N loading (mmol | — | 3.9 | 3.5 | 3.7 | 3.70 |
| per g fiber)^{d} | | | | | |
| Br loading (mmol | — | — | 0.12 | 0.14 | 0.20 |
| per g fiber)^{d} | | | | | |

^{a}Determined by nitrogen physisorption experiments at 77K.
^{b}Determined by the BJH method.
^{c}Measured by TGA at 35° C. in a 10% CO₂ atmosphere (balanced with N₂).
^{d}Determined by elemental analysis.

### Example 15. Batch Reaction of Propylene Carbonate with Butylamine, Hexylamine, Aniline, and 4-aminophenol in the Presence OF [HF-PAI/APS/3-Br-Prop]

The hydroxyalkylation reaction of aliphatic and aromatic amines with cyclic carbonate in the presence of [HF-PAI/APS/3-Br-prop] was carried out in a 100 mL high pressure stirred reactor operating in batch mode. The general procedure was performed as follows: 1) 0.3 g of the catalyst [HF-PAI/APS/3-Br-Prop] was added to the mixture of starting material. The mixture contained 6 mL of propylene carbonate and 1 mL of aromatic or aliphatic amines, (aniline, butylamine, hexylamine, and 4-aminophenol) under the reaction conditions of 60 PSI nitrogen, and 140° C. at different reaction times of 2, 4 and 8 hours. Schemes 7 and 8 show the reaction route of the aliphatic amines (butylamine and hexylamine) with propylene carbonate.

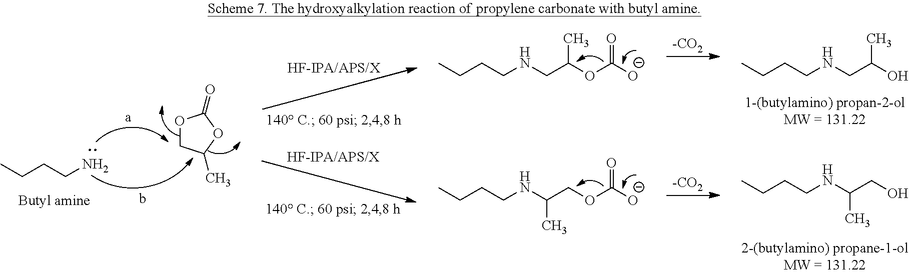

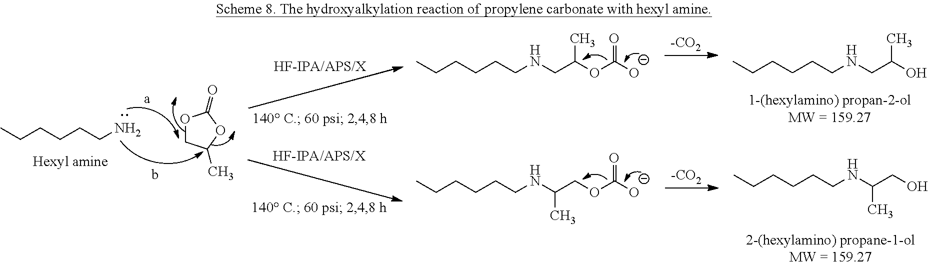

### Example 16. Batch Reaction of Propylene Carbonate with Butylamine, Hexylamine, Aniline, and 4-Aminophenol in the Presence of [HF-PAI/APS/Na-2-Br]

As mentioned in the previous example, the direct hydroxyalkylation reaction of amines and the nucleophilic ring opening of cyclic carbonate were performed in a 100 mL high pressure stirred reactor operating in batch mode. The procedure was performed by adding 1 cm³ of amine to 6 cm³ of propylene carbonate, the mixture and 100 mg of the [HF-PAI/APS/Na-2-Br] were introduced to the high pressure stirred reactor vessel.

The reaction was performed under 60 PSI N2 and at 140° C. for three different reaction times of 2, 4, and 8 hours. Schemes 9 and 10 illustrate the reaction mechanisms of the aromatic amines (aniline and 4-aminophenol) with PC in the presence of [HF-PAI/APS/Na-2-Br], [HF-PAI/APS/Na-2-Br], and [HF-PAI/APS/F]. The reactions were performed under the same reaction conditions.

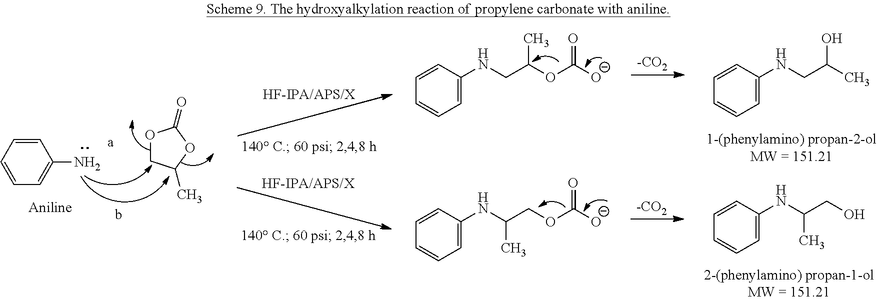

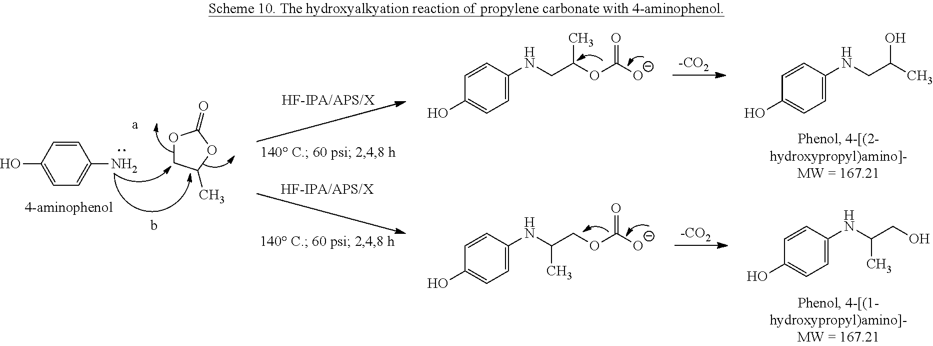

### Example 17. Batch Reaction of Propylene Carbonate with Butyl Amine, Hexylamine, Aniline, and 4-Aminophenole in the Presence of [HF-PAI/APS/F]

The hydroxyalkylation reaction of the aromatic amines (aniline and 4-aminophenole), and aliphatic amines (butylamine and hexylamine) with propylene carbonate was performed in a 100 cm³ high pressure stirred reactor. The reaction was conducted by adding 1 cm³ of amine to 6 mL of propylene carbonate to the reactor vessel, then 100 mg of the multifunctional organocatalysts [HF-PAI/APS/F], was added to the mixture. Schemes 7-10 illustrate the reaction mechanisms of both aliphatic and aromatic amines with propylene carbonate, respectively. The conditions of the reaction were 60 PSI N2, 140° C., at 2, 4, and 8 hours. The products and starting materials were analyzed on an Agilent 19091S-4 33 gas chromatograph (GC) equipped with mass spectroscopy (MS). Beyond GC-MS analysis, the products were determined by using ¹H nuclear magnetic resonance (NMR) and ¹³C NMR at room temperature with a Bruker-DRX 400 MHz spectrometer.

### Example 18. Cyclic Reaction of [HF-PAI/APS/3-Br-Prop] and [HF-PAI/APS/F]

Cyclic reaction experiments were performed by drying the fiber under vacuum oven at 80° C. for 2 hours to remove the permanent compounds, such as products or reactants. The fiber catalysts of [HF-PAI/APS/3-Br-Prop] and [HF-PAI/APS/F] were tested for three cycles to determine the degree of reuse and the extent of leaching of oxides from the fibers. The catalyst activity was determined, and the results were compared with fresh materials.

### Example 19. Physical and Chemical Characterization of the Catalysts of Examples 10-18

A variety of techniques have been used herein to characterize the synthesized catalysts and to demonstrate that the grafting with aminosilane and the immobilizing of bromine and fluorine were successful. The characterization results confirmed that the species, —OH, —NH, Br⁻, and F⁻ were covalently attached to the highly bonded porous cell walls of a hollow fiber surface; thus, a set of structural properties and catalytic fiber test results provided useful information to gain insight into the catalytic fixation of multifunction organocatalysts and their performance in hydroxyalkylation reactions. The process of characterization was applied on [HF-PAI/APS/3-Br-Prop], [HF-PAI/APS/Na-2-Br], and [PAI/APS/F]. Also, [HF-PAI (bare fiber)] and [HF-PAI/APS] were tested as well. The results of the textural properties are listed in Table 3.

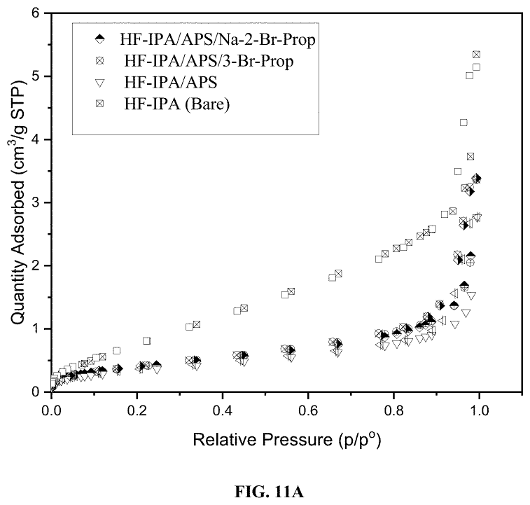

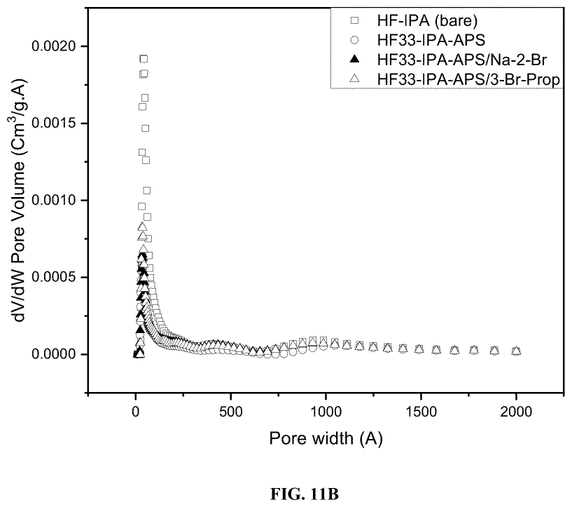

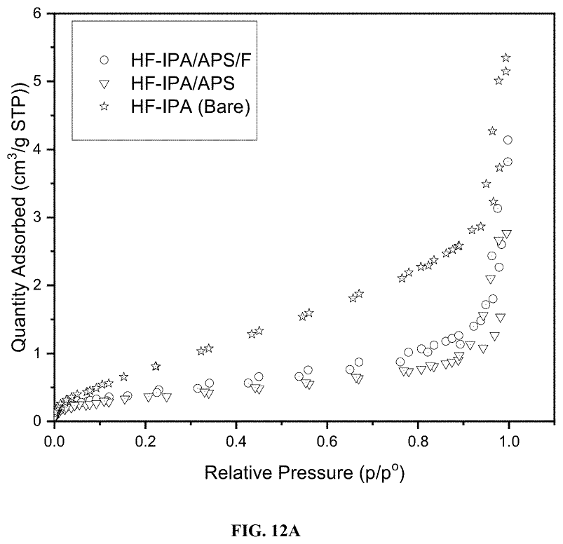

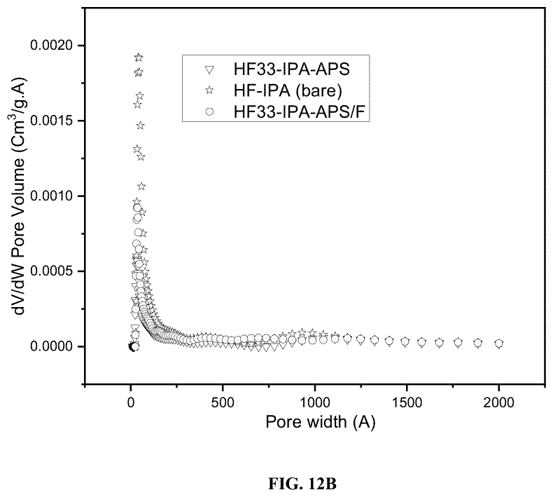

The Brunauer-Emmett-Teller (BET) surface area analysis and Barrett-Joyner-Halenda (BJH) pore size and volume analyses (FIGS. 11A, 11B, 12A, and 12B) demonstrate that the bare fibers HFs-PAI had the largest surface area and pore volume: 57.54 m²/g, 0.27 cm³/g, respectively. The pore size distribution was derived from the DFT method using the desorption branch of the N₂ isotherm. The surface area and pore volume were decreased either after the grafting with APS or after the immobilization of bromine or fluorine. The increased value could be due to the vacant sites on the bare fibers, the rearrangement of the polymer chain, and/or successful grafting and immobilizing of both APS and halogen into the bare PAIHFs.

The results illustrated that the post-treatment of the fibers makes the pore size small, especially towards the surface of the fibers, which might be related to the swelling of the polymer chains. Furthermore, the analysis of nitrogen adsorption-desorption isotherms for the HFs-PAI before and after grafting, also the immobilizing process of APS and halogens, respectively, are type IV. The pore diameter of the fiber was between 2 and 50 nm, which is the range of a mesopore according to the IUPAC.

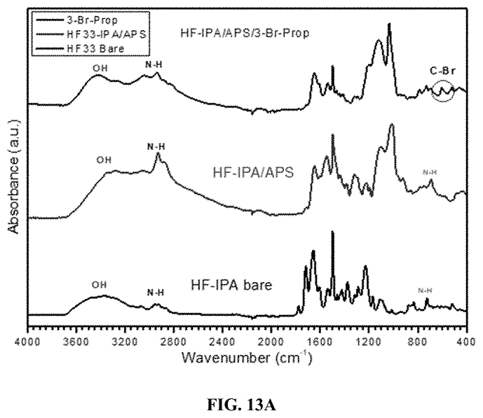

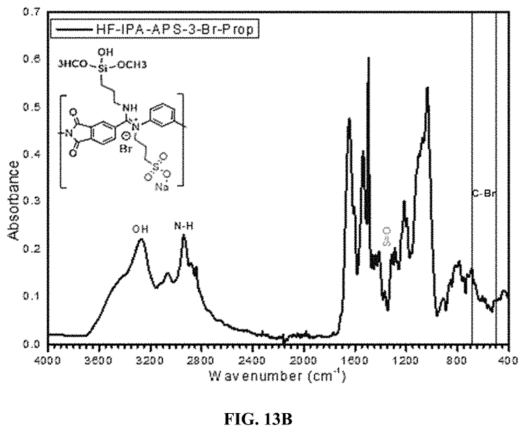

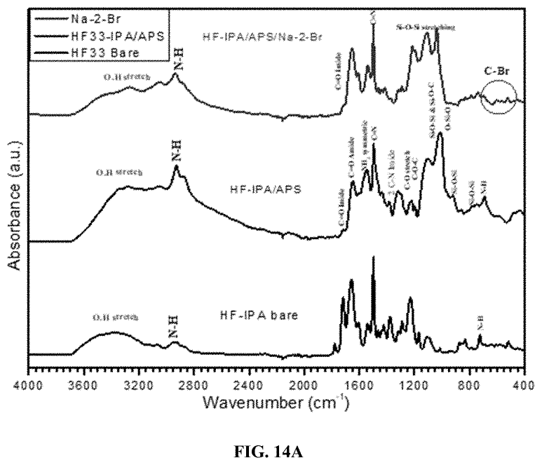

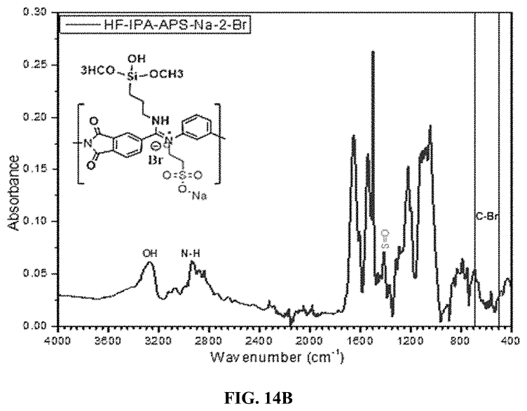

The Thermogravimetric Analysis (TGA) results in Table 3 indicated that the capacity of CO₂ over the fibers decreased after the grafting with APS and halogens which was helpful for the hydroxyalkylation reaction of the amines and prevented the occurrence of the reverse reaction, pushing the reaction towards the formation of products. For further confirmation, Fourier-Transform Infrared Spectroscopy (FTIR) was used to corroborate the effective grafting of APS, Br⁻, R⁻ into the polymer chains, including [HFs-PAI], [HFs-PAI/Aps], [HFs-PAI/Aps/3-Br-Prop], [HFs-PAI/Aps/Na-2-Br], and [HFs-PAI/Aps/F]. FTIR spectra of these cases are shown in FIGS. 13A, 13B, 14A, and 14B. The clear signals at 3000-3500 cm⁻¹ are related to the vibration of O—H hydrogen bonds in SiO—H attributed to APS. The signals at 3416-3468 cm⁻¹ are attributed to N—H that assigned to APS and polymer backbone. The stretched signals at 515-690 cm⁻¹ are attributed to the C—Br that ascribed to the functionalization of the polymer with bromide, and the signals at 730 cm⁻¹ and 1619 cm⁻¹ are related to Si—O bonds and conjugated C=N vibration that came from the cyclic system (imidazole), respectively.

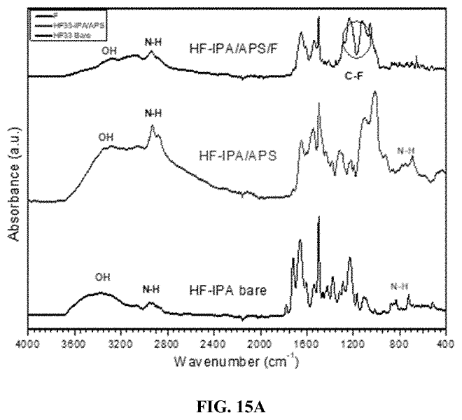

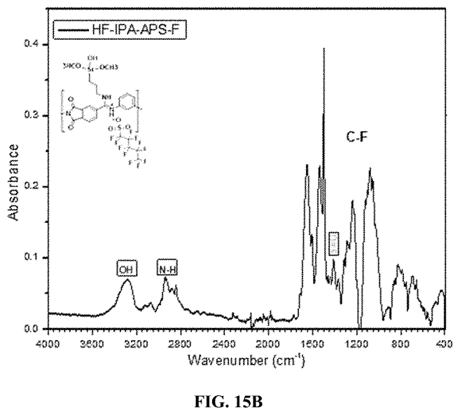

Furthermore, S═O spectra that attributed to the sulfur trioxide group (SO³⁻) was at 1380 cm⁻¹ to 1380 cm⁻¹ as indicated in FIG. 14B. In addition, the C—F bond spectra at the range of 1000-1400 cm⁻¹ that attributed to the fluorine source, which was nonafluorobutane-1-sulfonic acid, see FIG. 15B. Therefore, all these clarifications confirmed that the grafting and immobilization were successful on the body of the polymer, and it achieved the desired multifunction organocatalysts.

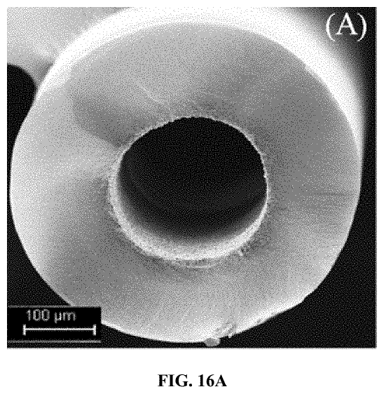

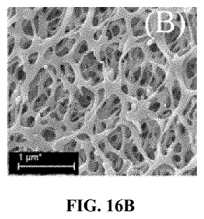

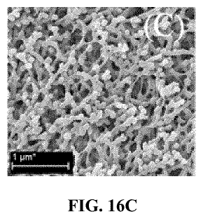

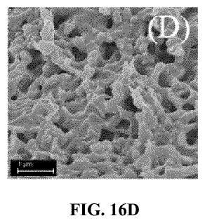

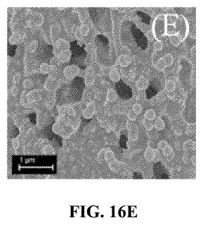

For more emphasis on the efficiency of the catalysts, the catalysts were tested by a scanning electron microscope (SEM) prior to and after the reaction. FIGS. 16A, 16B, 16C, 16D, and 16E show the SEM images that confirmed the fiber outer pore morphology was a little bit collapsed during post-treatment processing and a highly porous state was not maintained after APS grafting, and Br⁻ and F⁻ immobilization, FIG. 16C. However, the porous network of HF-PAI/APS/X was still retained, FIG. 16D. Furthermore, as can be seen from FIG. 16E, the fiber surface layer showed moderate porosity after the reaction, which indicates that there was a strong bonding between the Br⁻ & F⁻ and polymer matrix.

Some minor changes were observed on the surface of catalysts; however, the catalysts still maintained their efficiency, which confirms that the catalysts had a high bonding strength. This made them reusable without losing their catalytic efficiency, as can be clearly seen in FIG. 16C.

### Example 20. Effects of HF-PAI/APS/Na-2-Br and HF-PAI/APS/3-Br-Prop on the Course of the Hydroxyalkylation Reaction of Butylamine and Hexylamine with Propylene Carbonate

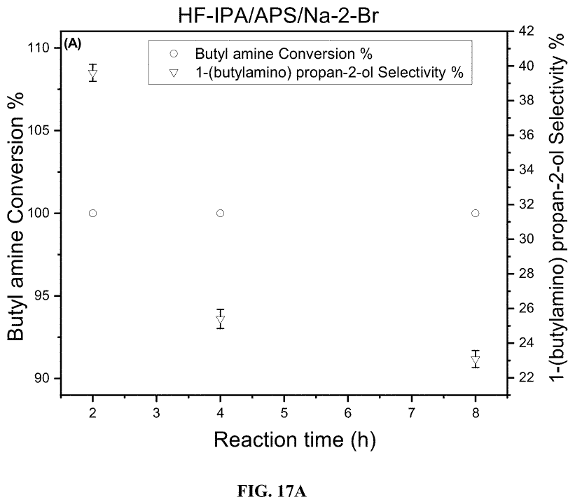

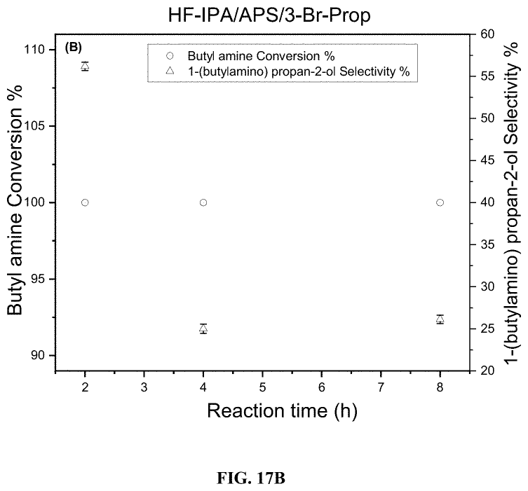

It was determined whether the designed catalysts materials are active for the hydroxyalkylation reaction of aliphatic and aromatic amines with propylene carbonate. For confirmation, the catalysts were tested in a 100 mL high pressure stirred reactor operating in batch mode. The catalytic performance of HF-PAI/APS/Na-2-Br and HF-PAI/APS/3-Br-Prop was assessed for the hydroxyalkylation reaction of butylamine with propylene carbonate towards the synthesis of an aminoalcohols, as presented in Scheme 7. This reaction mechanism has two pathways, a and b. Pathway a lead to the formation of the major product, 1-(butylamino) propan-2-ol, which is the most productive. Pathway b gave the byproduct, which was the least productive, 2-(butylamino) propane-1-ol. The respective structures of the products were determined by GC-MS and NMR. As seen in FIGS. 17A and 17B, the conversion of butylamine was 100% in the presence of both catalysts at all of the reaction times of 2, 4, and 8 hours. These results confirm that the catalysts had a high catalytic ability to convert amines under moderate reaction conditions.

Regarding activity, efficiency, and ease of use in terms of their separation from the products, all these properties marked the catalysts as very important for the hydroxyalkylation reaction, so the present catalysts should be taken into consideration as an alternative to homogeneous catalysts. On the other hand, the selectivity and the yield of 1-(butylamino) propan-2-ol was at 40% in 2 hours, and slightly decreased to 23% at 8 hours, in the presence of HF-PAI/APS/Na-2-Br; while, under the HF-PAI/APS/3-Br-Prop the selectivity was 56% at 2 hours, and dramatically decreased to about 26% at 4 and 8 hours. This behavior indicated that the hydroxyalkylation reaction of butylamine took place in a short time, and then the selectivity of the products was decreased by the increase of the reaction time because the reactions usually slowed down over time due to the depletion of the reactants. Thus, the reaction gave the highest selectivity of the product at the first two hours of the reaction time, as shown in FIGS. 17A and 17B.

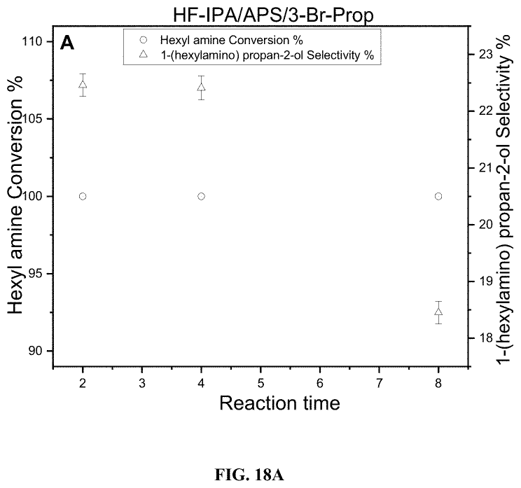

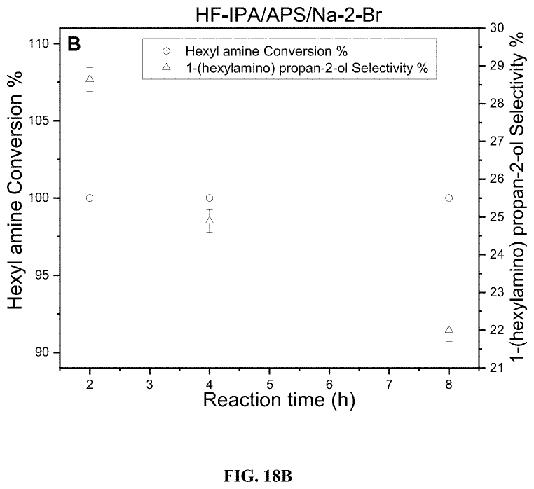

In addition, the effect of reaction time on hexamine conversion and product selectivity has been studied, and the obtained results (FIGS. 18A and 18B) indicated that the hexamine conversion was 100% at all times of the reaction, which proves that the catalysts had a high potential to convert the aliphatic amines, and also activate the amines to insert into PC to open ring via nucleophilic attack for the formation of a covalent bond between carbon and nitrogen to produce amino alcohols. As for the selectivity of the products, the results showed that in the presence of [HF-PAI/APS/3-Br-Prop], FIG. 18A, the selectivity of the 1-(hexylamino) propan-2-ol was 22.5% at 2 and 8 hours and slightly decreased to −18% at 8 hours. While, in the presence of [HF-PAI/APS/Na-2-Br], FIG. 18B, the selectivity of 1-(hexylamino) propan-2-ol was enhanced a little bit to 29% at 2 hours and then decreased to 21.9% at 8 hours. Thus, the highest selectivity of products under both catalysts was achieved at 2 hours. Although the selectivity was not high, the reaction occurred in a short time and achieved a high conversion of amines at the same time as mentioned above. The respective structures of the products were determined by GC-MS, and ¹H and ¹³C NMR.

### Example 21. Effects of HF-PAI/APS/Na-2-Br and HF-PAI/APS/3-Br-Prop on the Course of the Hydroxyalkylation Reaction of Aniline and 4-Aminophenol with Propylene Carbonate

According to the reaction mechanism of the aniline with PC (Scheme 9), the reaction followed two approaches. Approach a resulted in the formation of the major product 1-(phenylamino) propan-2-ol, while approach b led to producing the minor product, 2-(phenylamino) propan-1-ol. The formation of these products depended on where the amine group was attacked the propylene carbonate ring. The attack of the amine group opened the stable propylene carbonate ring under mild reaction conditions, and the outcome was based on the catalytic activity.

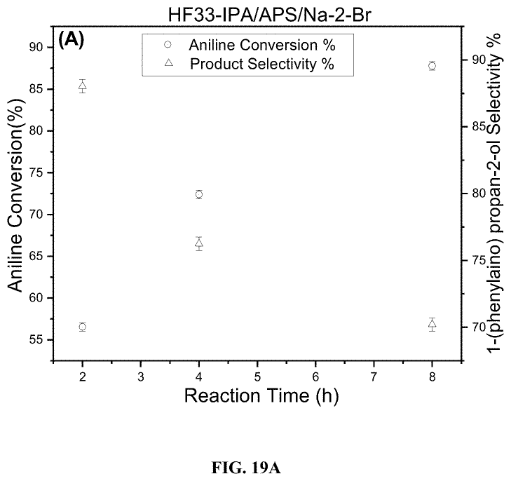

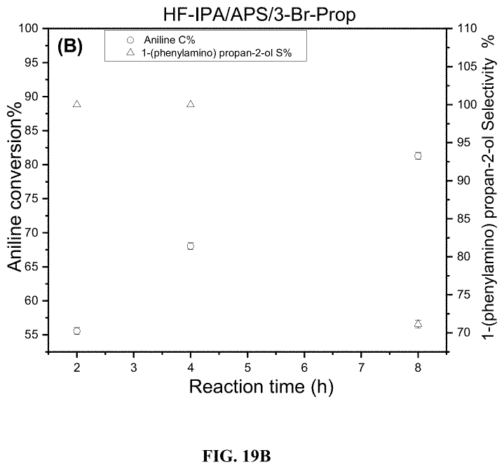

FIGS. 19A and 19B present the conversion of aniline in the presence of [HF-PAI/APS/Na-2-Br] and [HF-PAI/APS/3-Br-Prop]; the conversion was 88% and 97.5 at 8 hours, respectively. It is evident that the conversion was increased by increasing the reaction time, and the reaction is characterized by achieving a high conversion even at 2 hours. Meanwhile, in the presence of [HF-PAI/APS/Na-2-Br] the selectivity of product 1-(phenylamino) propan-2-ol was 87% at 2 hours and slightly decreased to 70% at 8 hours, while only forming 2.81% of byproduct at 8 hours. However, under [HF-PAI/APS/3-Br-Prop] the selectivity of 1-(phenylamino) propan-2-ol was reached (up to 100%) at 2 and 4 hours, and slightly decreased to about 70% at 8 hours. Thus, the results confirmed that the hydroxylation reaction of aniline with PC was very successful in the presence of both catalysts, which indicated that the immobilized catalysts with multi active sites is an excellent approach to generate aminoalcohols under moderate reaction conditions, therefore the existence of the bromide and other species (—NH & —OH) increased the product selectivity and suppress undesired side reactions. The respective structures of the products were determined by GC-MS, ¹H NMR and ¹³C NMR.

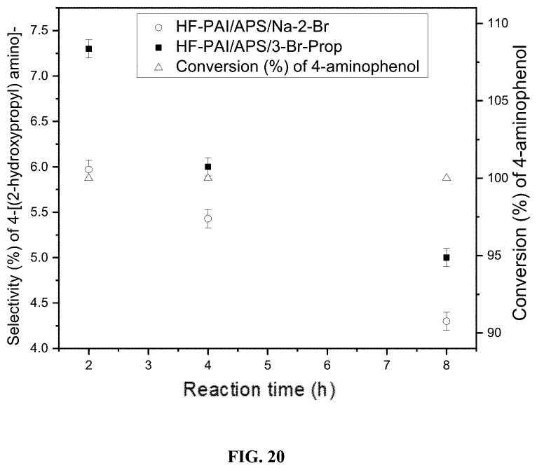

The hydroxyalkylation of aniline with PC gave impressive results. The hydroxyalkylation reaction of 4-aminophenol with PC under the same reaction conditions gave different results. As is evident from FIG. 20, the selectivity of the product was about 8% at 2 hours and slightly decreased to around 6% at 4 hours and 5% 8 hours. The decrease in selectivity could be due to the effect of hydroxyl group that attached at the other end of the benzene ring, or as a result of having more than one product at the same time, which has a negative impact on pushing the reaction towards the formation of the main product 4-[(2-hydroxypropyl) amino]-. The reaction gave a high conversion of the 4-aminophenol, which indicates that the activity of the catalysts was high. The conversion of 4-AP was reached (up to 100%) at all times of the reaction, see FIG. 20.

### Example 22. Effects of HF-PAI/APS/F on the Course of the Hydroxyalkylation Reaction of Aniline and 4-Aminophenole with Propylene Carbonate

The catalytic activities of HF-PAI/APS/F on the hydroxyalkylation reaction of aniline with propylene carbonate (PC) was carried out in a 100 cm³ high pressure stirred reactor operating in the batch mode. The ratio of amine to (PC) was 1:6 cm³, respectively, and the reaction conditions were 140° C., 60 PSI N2, free solvent, and at 2, 4, and 8 hours. All reactions were stopped after 8 hours and analyzed by GC-MS and NMR.

Scheme 9 shows the proposed reaction mechanisms among cyclic carbonates (PC) and aromatic amines (aniline). The reaction mechanisms illustrate that the amine group attacked the stable propylene carbonate ring in two positions, the first attack was on the carbon next to the carbon that holding the methyl group which led to forming the major product route (a), 1-(phenylamino) propane-2-ol; while the second attack was on the carbon that holding the methyl group which formed the minor product as could be seen in route (b), 2-(phenylamino) propane-1-ol.

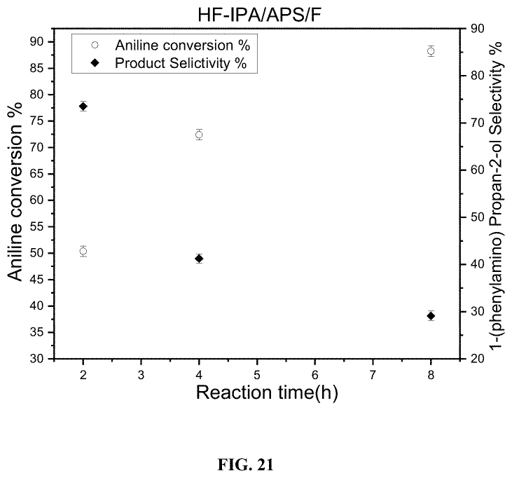

The produced products and aniline conversion are presented in FIG. 21. Aniline conversion reached 88%, while the selectivity of 1-(phenylamino) propan-2-ol at 2 hours was 73%. Product selectivity was decreased with the increasing of the time from 2 hours to 8 hours. Thus, the decrease in the selectivity of 1-(phenylamino) propan-2-ol from 73% at 2 hours to 29% at 8 hours could be due to the behavior of the halogen, where the fluorine is less stable and more reactive. In the presence of trifunctional organocatalysts, the highest conversion of aniline (100%) and product selectivity (73%) were achieved at 140° C. and 2 hours, thus this great achievement makes the present catalyst more reliable for use in hydroxyalkylation reactions of amines with cyclic carbonates. The reaction conversion and the amounts of products were determined by GC-MS analyses, and the structure of the products was determined by GC-MS, ¹H NMR, and ¹³C NMR.

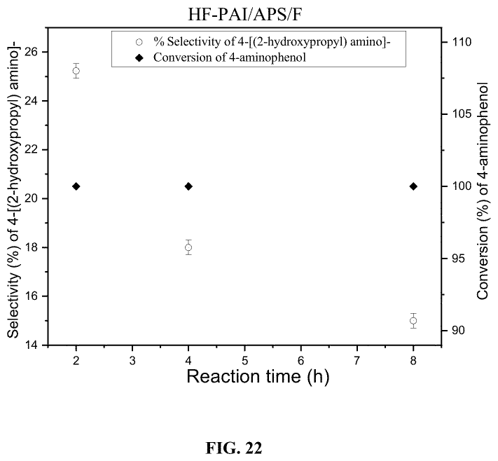

With an understanding of the mechanism for hydroxyalkylation of aniline, the hydroxyalkylation of 4-aminophenol with PC in the presence of HF-PAI/APS/F was also studied. The proposed reaction mechanism (Scheme 10) reported two basic products. The major product was 4-[(2-hydroxypropyl) amino]—route a, whereas the minor product was 4-[(1-hydroxypropyl) amino]—route b. According to the results presented in FIG. 22, the conversion of 4-AP was reached (up to 100%) at all times of the reaction, however the selectivity of the major product was 29% at 2 hours and then decreased to around 17.5% at 4 hours and 16% at 8-hours. This change in selectivity may be a result of the influence of subsidiary products, since a variety of by-products were detected by GC-MS, and this number of by-products may have a negative effect on the selectivity of the main product.

### Example 23. Effects of HF-PAI/APS/F on the Course of the Hydroxyalkylation Reaction of Butylamine and Hexylamine with Propylene Carbonate

To demonstrate the synthetic utility of the present catalysts toward the hydroxyalkylation reaction of amines with PC in a wider scope, the hydroxyalkylation reaction of aliphatic amines with PC in the presence of HF-PAI/APS/F was performed. The proposed mechanism (Scheme 7) for this reaction was summarized in the following reaction routes. Route a presented the formation of, [1-(butylamino) propan-2-ol], which was the most productive, while approach b presented the byproduct's formation route, [2-(methylamino) propane-1-ol], which was the least productive. For all 2, 4, and 8 hour reaction times, the butylamine conversion was 100%. This high conversion confirms that the HF-PAI/APS/F had a high catalytic ability to convert amines under moderate reaction conditions. Without being bound by any particular theory, the efficiency of the catalyst for reaching high conversion could be due to the fluorine activity and its high electronegativity that pushed the reaction to convert the butylamine and inserted it into the ring of PC.

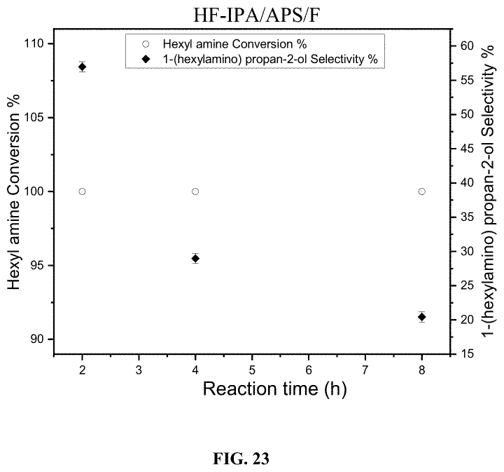

In addition to butylamine conversion, the selectivity of the product was also studied to investigate the catalyst activity toward the formation of the amino alcohol. The results of C % and S % are reported in FIG. 23. The selectivity and the yield of 1-(butylamine) propan-2-ol was reached 40% in 2 hours, and slightly decreased to 30.5% at 8 hours, which means the hydroxyalkylation reaction of butylamine in the presence of HF-PAI/APS/F, was accomplished in the first two hours of reaction.

Additionally, the effect of the reaction time on the conversion of hexylamine and the selectivity of the product has been investigated too. The reaction pathways (a) and (b) show the primary and secondary product, respectively (Scheme 8). The ability of the catalysts to convert the hexylamine, and its capacity to intercalate the reaction to open the propylene carbonate ring via nucleophilic attack for the formation of a covalent bond between carbon and nitrogen in the direction of aminoalcohols formation, was investigated. The conversion of hexylamine reached up to 100% at all times of the reaction (FIG. 23).

### Example 24. Reuse of Hollow Fiber Catalysts

To estimate the stability of the catalysts after reaction, three runs of experiment recycling were performed in which the used hollow fiber catalysts were first separated from the products and dried in a vacuum oven to sufficiently remove the remained products or reactants from the fiber pores, and then reused in aniline hydroxyalkylation reaction with PC under the same reaction conditions that applied with the original one. Results are shown in Table 4.

TABLE 4
Reuse of HF-PAI/APS/3-Br-Prop catalysts for aniline
hydroxyalkylation reaction with PC at 2, 4, and 8 hours.

| | | HF-PAI/APS/3-Br-Prop | |
|---|---|---|---|
| Reaction | Reaction time | | 1-(phenylamino)- |
| Cycle | (h) | Aniline Con. (%) | 2-ol, Sel. (%) |
| 1st | 2 | 55.56 | 100 |
| | 4 | 68.03 | 100 |
| | 8 | 81.3 | 82 |
| 2nd | 2 | 56.03 | 100 |
| | 4 | 67.1 | 100 |
| | 8 | 80.43 | 80 |
| 3rd | 2 | 53.008 | 71.14 |
| | 4 | 67.5 | 71.5 |
| | 8 | 76.22 | 65 |

Reaction conditions:
temperature: 140° C.;
1 mL aniline;
6 mL PC;
100 mg catalyst;
60 PSI N₂

Both the aniline conversion and the selectivity of 1-(phenylamino)-2-ol were similar to those obtained with fresh hollow fiber catalysts. Without being bound by theory, the slight decrease in activity may be due to the loss of access to some active sites on the porous hollow fiber stimuli. These results illustrated that some swelling effects were still present in the HF-PAI/APS/3-Br-Prop, although the degree of swelling was decreased compared to the bare hollow fibers. HF-PAI/APS/3-Br-Prop had maintained its activity and resulted in the conversion of 81.3% aniline and about 71%, 1-(phenylamino)-2-ol selectivity. Therefore, the hollow fibers can be reused without much loss to the active sites of the catalyst.

In addition, the results of cyclic reaction of HF-PAI/APS/F showed that the catalyst had high stability, and no significant difference was observed between the three operations, see Table 5. Therefore, the catalyst preserved its catalytic activity and so it can be reused without much loss of the active sites of the catalyst.

TABLE 5
Cyclic reaction of HF-PAI/APS/F catalyst for aniline
hydroxyalkylation reaction with PC at 2, 4, and 8 hours.

| | | HF-PAI/APS/3-Br-Prop | |
|---|---|---|---|
| Reaction | Reaction time | | 1-(phenylamino)- |
| Cycle | (h) | Aniline Con. (%) | 2-ol, Sel. (%) |
| 1st | 2 | 50.37 | 73.56 |
| | 4 | 72.41 | 41.26 |
| | 8 | 88.25 | 29.12 |
| 2nd | 2 | 48.75 | 73.1 |
| | 4 | 71.43 | 40.6 |
| | 8 | 87.01 | 28.2 |
| 3rd | 2 | 45.05 | 71.9 |
| | 4 | 72.8 | 41 |
| | 8 | 88.1 | 28.5 |

Reaction conditions:
temperature: 140° C.;
1 mL aniline;
6 mL PC;
100 mg catalyst;
60 PSI N₂

Overall, the use of porous polymers support and immobilization of various homogeneous organocatalysts offers the catalyst designer different, and perhaps more, opportunities in the design of cooperative organocatalysts. Advantages of weak acid-base-halide trifunctional organocatalysts sites also relate to the stabilization of unstable substrates and/or products that would easily be converted to other products and can be used for all substrates soluble in water and also organic solvents. The present disclosure has described the development and application of a high-performance immobilized trifunctional amine/acid/halide organocatalysts for the efficient, one-step catalyst reaction of CO₂ cycloadditions and aminolysis of aliphatic and aromatic amines. Catalyst recycling without significant loss of activity or selectivity was demonstrated over 3 cycles. Catalysts according to the present disclosure may be used in a variety of reactions in the field of flow chemistry.

This written description uses examples to illustrate the present disclosure, including the best mode, and also to enable any person skilled in the art to practice the disclosure, including making and using any compositions or systems and performing any incorporated methods. The patentable scope of the disclosure is defined by the claims, and may include other examples that occur to those skilled in the art. Such other examples are intended to be within the scope of the claims if they have elements that do not differ from the literal language of the claims, or if they include equivalent elements with insubstantial differences from the literal language of the claims.

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where an invention or a portion thereof is defined with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As used herein, the term “about” means plus or minus 10% of the value.

What is claimed is:

1. An organocatalyst comprising:
a porous hollow fiber; and
at least two functional groups covalently bonded to the porous hollow fiber;
wherein the organocatalyst is free of metals.

2. The organocatalyst of claim 1, wherein at least one functional group of the at least two functional groups covalently bonded to the porous hollow fiber is a capturing functional group and wherein at least one functional group of the at least two functional groups covalently bonded to the porous hollow fiber is a reactive functional group.

3. The organocatalyst of claim 1, wherein the organocatalyst comprises at least three functional groups.

4. The organocatalyst of claim 1, wherein at least one functional group of the at least two functional groups covalently bonded to the porous hollow fiber is bonded covalently in a pore of the porous hollow fiber.

5. The organocatalyst of claim 1, wherein at least one functional group of the at least two functional groups covalently bonded to the porous hollow fiber is bonded covalently on a surface of the porous hollow fiber.

6. The organocatalyst of claim 1, wherein at least one functional group of the at least two functional groups covalently bonded to the porous hollow fiber imparts more than one functionality to the organocatalyst.

7. The organocatalyst of claim 1, wherein at least one functional group of the at least two functional groups covalently bonded to the porous hollow fiber imparts a functionality selected from the group consisting of hydrogen donation, electron donation, electron withdrawal, acidity, basicity, nucleophilicity, electrophilicity, enantioselectivity, and combinations thereof.

8. The organocatalyst of claim 1, wherein at least one functional group of the at least two functional groups covalently bonded to the porous hollow fiber comprises a functional group selected from the group consisting of halogen, fluorine, chlorine, bromine, iodine, haloalkyl, hydroxyl, sulfur, sulfur trioxide, oxygen, amine, amide, carbonyl, carboxyl, ester, ether, alkane, alkene, alkyne, chiral cyclophosphazane, phosphorus amide, silane, aminosilane, and combinations thereof.

9. The organocatalyst of claim 1, wherein the organocatalyst comprises a halogen group and an aminosilane group.

10. A chemical reactor comprising the organocatalyst of claim 1.

11. A process for producing an organocatalyst comprising:
a porous hollow fiber; and
at least two functional groups covalently bonded to the porous hollow fiber;
wherein the organocatalyst is free of metals, the process comprising:
spinning a porous hollow fiber;
grafting a first functional group to the porous hollow fiber; and
grafting a second functional group to the porous hollow fiber.

12. The process of claim 11, wherein the process step of spinning the porous hollow fiber comprises spinning the porous hollow fiber with a technique selected from the group consisting of dry-jet, wet-quench spinning, melt spinning, dry spinning, wet spinning, and combinations thereof.

13. The process of claim 11, wherein at least one of the process steps of grafting a first functional group to the porous hollow fiber and grafting a second functional group to the porous hollow fiber comprises covalently bonding a functional group to the porous hollow fiber.

14. A process for reacting chemicals, the process comprising:
introducing a first reactant to an organocatalyst comprising:
a porous hollow fiber; and
at least two functional groups covalently bonded to the porous hollow fiber;
wherein the organocatalyst is free of metals;
introducing a second reactant to the organocatalyst; and
reacting the first reactant and the second reactant.

15. The process of claim 14, wherein the process is a continuous flow process.

16. The process of claim 14, wherein the process step of reacting the first reactant and the second reactant comprises heating the organocatalyst.

17. The process of claim 14, wherein the process step of reacting the first reactant and the second reactant comprises a heterogeneous reaction.

18. The process of claim 14, wherein the first reactant comprises a reactive compound selected from the group consisting of H₂O, CO₂, SO₂, SOₓ, NO₂, NOₓ, CO, olefins, and combinations thereof.

19. The process of claim 14, wherein the second reactant comprises a reactive gas or reactive liquid.

20. The process of claim 14, wherein the organocatalyst comprises a halogen group and an aminosilane group.
